(19) United States
(12) Patent Application Publication
(10) Pub. No.: US 2026/0199587 A1
(43) Pub. Date: Jul. 16, 2026

(54) COMMUNICATION SYSTEMS AND METHODS FOR NEGATIVE PRESSURE WOUND THERAPY DEVICES

(71) Applicant: T.J.Smith and Nephew,Limited, Hull (GB)

(72) Inventors: Baha Badran, Enniscorthy (IE); David Michael Elder, Hull (GB); Felix Clarence Quintanar, Hull (GB); Steafán Sherlock, Dublin (IE); Jinsong Song, Enniscorthy (IE)

(21) Appl. No.: 19/561,991

(22) Filed: Mar. 10, 2026

Related U.S. Application Data
(63) Continuation of application No. 18/287,856, filed on Apr. 20, 2022, now Pat. No. 12,582,763.
(60) Provisional application No. 63/177,800, filed on Apr. 21, 2021.

(51) Int. Cl. A61M 1/00

(52) U.S. Cl. CPC A61M 1/982; A61M 2205/14; A61M 2205/3569; A61M 2205/3584; A61M 2205/6027

(57) ABSTRACT

A negative pressure wound therapy device can include one or more antennas and communication circuitry. Various communication protocols can be supported, including short, medium, and long range communication protocols. Depending on the environment and application, various data related to the status of the negative pressure wound therapy device or any of the accessories can be transmitted using the one or more communication protocols. 62804979

### CROSS-REFERENCE TO RELATED APPLICATIONS

This application is a continuation of U.S. application Ser. No. 18/287,856, filed Oct. 20, 2023, which is a U.S. national stage application of International Patent Application No. PCT/EP 2022/060464, filed Apr. 20, 2022 and published as WO 2022/223646, which claims priority to U.S. Provisional Application No. 63/177,800, filed Apr. 21, 2021, each of which is hereby incorporated by reference in its entirety.

### TECHNICAL FIELD

Embodiments described herein relate to apparatuses, systems, and methods for the treatment of wounds, for example using dressings in combination with negative pressure wound therapy.

### DESCRIPTION OF THE RELATED ART

Many different types of wound dressings are known for aiding in the healing process of a human or animal. These different types of wound dressings include many different types of materials and layers, for example, gauze, pads, foam pads or multi-layer wound dressings. Topical negative pressure (TNP) therapy, sometimes referred to as vacuum assisted closure, negative pressure wound therapy, or reduced pressure wound therapy, is widely recognized as a beneficial mechanism for improving the healing rate of a wound. Such therapy is applicable to a broad range of wounds such as incisional wounds, open wounds, and abdominal wounds or the like. TNP therapy assists in the closure and healing of wounds by reducing tissue edema, encouraging blood flow, stimulating the formation of granulation tissue, removing excess exudates and may reduce bacterial load. Thus, reducing infection to the wound. Furthermore, TNP therapy permits less outside disturbance of the wound and promotes more rapid healing.

### SUMMARY

A negative pressure wound therapy device can include a housing. The device can include a negative pressure source supported by the housing. The negative pressure source can be configured to provide negative pressure to a wound covered by a wound dressing. The device can include an electronic control circuitry supported by the housing. The electronic control circuitry can be configured to operate the negative pressure source. The electronic control circuitry can include a first antenna positioned in a top portion of the housing or a side portion of the housing. The first antenna can be configured to facilitate wireless communications using a first communication protocol. The electronic circuitry can include a second antenna positioned in a different location from the first antenna. The second antenna can be configured to facilitate wireless communications using the first communication protocol. The electronic circuitry can include a third antenna configured to facilitate wireless communications using a second communication protocol that is different from the first communication protocol.

The negative pressure wound therapy device of any of the preceding paragraphs and/or any of the devices, systems, or apparatuses disclosed herein can include one or more of the following features. The electronic control circuitry can be configured to transmit, using the first antenna, to a remote computing device pairing information for pairing the remote computing device with the negative pressure wound therapy device to allow exchange of data between the remote computing device and the negative pressure wound therapy device using the second communication protocol. The second communication protocol can permit communication over a longer distance than the first communication protocol. The first communication protocol can include a near-field protocol. The second communication protocol can include Bluetooth or Bluetooth low energy (BLE) protocol. The electronic control circuitry can be configured to non-simultaneously transmit or receive data using the first or second antenna. The second antenna can be configured to obtain canister data from a canister removably attached to the housing. The canister data can include one or more of fill status of the canister, an indication that the canister is full, identification data, configuration data, date of manufacture of the canister, or date of first use of the canister. The electronic control circuitry can be configured to determine that the canister is connected to housing responsive to receiving the canister data. The electronic control circuitry can be configured to disallow activation of the negative pressure source responsive to determining that the canister is not connected to the housing.

The negative pressure wound therapy device of any of the preceding paragraphs and/or any of the devices, systems, or apparatuses disclosed herein can include one or more of the following features. The electronic control circuitry can include a first transceiver connected to the first and third antennas and a second transceiver connected to the second antenna. The electronic control circuitry can include a first matching circuitry interposed between the first transceiver and the first antenna. The first matching circuitry can be configured to match impedance of the first antenna with a first input impedance of the first transceiver. The electronic control circuitry can include a second matching circuitry interposed between the first transceiver and the second antenna. The second matching circuitry can be configured to match impedance of the second antenna with a second input impedance of the first transceiver. The electronic control circuitry can include a third matching circuitry interposed between the second transceiver and the third antenna. The third matching circuitry can be configured to match impedance of the third antenna with an input impedance of the second transceiver. The first matching circuitry can include at least one capacitor and at least one resistor. The third matching circuitry can include at least one capacitor, at least one inductor, and at least one resistor. The first antenna can be positioned in the top portion of the housing, and the second antenna can be positioned in a bottom portion of the housing. The canister can be configured to be removably supported by the bottom portion of the housing. The third antenna can be positioned in the top portion of the housing proximate to the first antenna. The first and third antennas can be positioned on a circuit board. The third antenna can include a planar inverted-F antenna.

A negative pressure wound therapy device can include a housing. The device can include a negative pressure source supported by the housing. The negative pressure source can be configured to provide negative pressure to a wound covered by a wound dressing. The device can include an electronic control circuitry supported by the housing. The electronic control circuitry can be configured to operate the negative pressure source. The electronic control circuitry can include a cellular antenna configured to facilitate wireless communications using long term evolution (LTE) communication protocol. The electronic control circuitry can include a global positioning system (GPS) antenna. The electronic control circuitry can include a transceiver configured to transmit or receive data using the cellular antenna and the GPS antenna. The electronic control circuitry can include a matching circuitry interposed between the cellular antenna and the transceiver. The matching circuitry can be configured to match impedance of the cellular antenna with an input impedance of the transceiver.

The negative pressure wound therapy device of any of the preceding paragraphs and/or any of the devices, systems, or apparatuses disclosed herein can include one or more of the following features. The matching circuitry can include a T-network or an L-network with a plurality of capacitors. The cellular antenna can be located in a bottom portion of the housing. The GPS antenna can be positioned in a top portion of the housing or a side portion of the housing. The transceiver is supported by a first printed circuit board (PCB) supported by the housing. The cellular antenna can be supported by a second PCB mounted to the first PCB. The second PCB can be electrically connected to the first PCB by a single connector of the first PCB. The single connector can provide mechanical support for the second PCB.

The negative pressure wound therapy device of any of the preceding paragraphs and/or any of the devices, systems, or apparatuses disclosed herein can include one or more of the following features. The device can include a first power source configured to provide power at least to the negative pressure source and a second power source configured to provide power at least to the transceiver. The transceiver can be configured to transmit location information using LTE or 5G communication protocol. The transceiver can be configured to transmit location information using the LTE or 5G communication protocol when the first power source does not provide power to the negative pressure source or any other component of the negative pressure wound therapy device. The transceiver can be configured to transmit location information using the LTE communication protocol that includes LTE Cat M1. The electronic control circuitry can be configured to disable the cellular antenna responsive to detecting that the negative pressure wound therapy device is being carried on an aircraft. The electronic control circuitry can be configured to detect that the negative pressure wound therapy device is being carried on an aircraft based on monitoring at least one of an altitude or vibration.

The negative pressure wound therapy device of any of the preceding paragraphs and/or any of the devices, systems, or apparatuses disclosed herein can include one or more of the following features. The GPS antenna can include an amplifier. The electronic control circuitry can include circuitry configured to provide power to the amplifier using the same connection of the GPS antenna on which data is communicated to the transceiver.

Disclosed herein are methods of operating a negative pressure wound therapy device of any of the preceding paragraphs and/or any of the devices, apparatuses, or systems disclosed herein.

Disclosed herein are kits that include the negative pressure wound therapy device of any of the preceding paragraphs and/or any of the devices, apparatuses, or systems disclosed herein and a canister and/or one or more wound dressings.

Any of the features, components, or details of any of the arrangements or embodiments disclosed in this application, including without limitation any of the apparatus embodiments and any of the negative pressure wound therapy embodiments disclosed herein, are interchangeably combinable with any other features, components, or details of any of the arrangements or embodiments disclosed herein to form new arrangements and embodiments.

### BRIEF DESCRIPTION OF THE DRAWINGS

FIG. 1A illustrates a negative pressure wound therapy system.

FIG. 1B illustrates another negative pressure wound therapy system.

FIG. 2A is an isometric view of a negative pressure wound therapy device and canister, showing the canister detached from the pump assembly of the device.

FIG. 2B is a back view of the negative pressure wound therapy device shown in FIG. 2A.

FIG. 2C illustrates a top surface of the negative pressure wound therapy device shown in FIG. 2A, showing a user interface.

FIG. 3 illustrates a schematic of a control system of a negative pressure wound therapy device.

FIG. 4 illustrates another negative pressure wound therapy system.

FIGS. 5A-5I illustrate exploded views of the negative pressure wound therapy device of FIG. 2A.

FIGS. 6A-6B illustrate schematics of electronic communications circuitry of the negative pressure wound therapy device of FIG. 2A.

FIGS. 7A-7C illustrate a communications assembly for cellular and global positioning system (GPS) communications.

FIGS. 8A-8B illustrate a top layer and bottom layer of a communications circuit board for cellular communications.

FIG. 9 illustrates a schematic of electronic communications circuitry for cellular communications.

FIGS. 10A-10B illustrate integration of a GPS antenna into a negative pressure wound therapy device.

### DETAILED DESCRIPTION

Embodiments disclosed herein relate to systems and methods of treating and/or monitoring a wound. Some embodiments of the negative pressure wound therapy devices disclosed herein can include a negative pressure source configured to be connected and/or fluidically coupled, via a fluid flow path, to a wound covered by a wound dressing and provide negative pressure to a wound.

Throughout this specification reference is made to a wound. The term wound is to be broadly construed and encompasses open and closed wounds in which skin is torn, cut or punctured or where trauma causes a contusion, or any other superficial or other conditions or imperfections on the skin of a patient or otherwise that benefit from pressure treatment. A wound is thus broadly defined as any damaged region of tissue where fluid may or may not be produced. Examples of such wounds include, but are not limited to, abdominal wounds or other large or incisional wounds, either as a result of surgery, trauma, sterniotomies, fasciotomies, or other conditions, dehisced wounds, acute wounds, chronic wounds, subacute and dehisced wounds, traumatic wounds, flaps and skin grafts, lacerations, abrasions, contusions, bums, diabetic ulcers, pressure ulcers, stoma, surgical wounds, trauma and venous ulcers or the like.

Embodiments of systems and methods disclosed herein can be used with topical negative pressure (“TNP”) or reduced pressure therapy systems. Briefly, negative pressure wound therapy assists in the closure and healing of many forms of “hard to heal” wounds by reducing tissue oedema, encouraging blood flow and granular tissue formation, or removing excess exudate and can reduce bacterial load (and thus infection risk). In addition, the therapy allows for less disturbance of a wound leading to more rapid healing. TNP therapy systems can also assist in the healing of surgically closed wounds by removing fluid. TNP therapy can help to stabilize the tissue in the apposed position of closure. A further beneficial use of TNP therapy can be found in grafts and flaps where removal of excess fluid is important and close proximity of the graft to tissue is required in order to ensure tissue viability.

As used herein, reduced or negative pressure levels, such as-X mmHg, represent pressure levels relative to normal ambient atmospheric pressure, which can correspond to 760 mmHg (or 1 atm, 29.93 inHg, 101.325 kPa, 14.696 psi, etc.). Accordingly, a negative pressure value of −X mmHg reflects pressure that is X mmHg below 760 mmHg or, in other words, a pressure of (760−X) mmHg. In addition, negative pressure that is “less” or “smaller” than X mmHg corresponds to pressure that is closer to atmospheric pressure (for example, −40 mmHg is less than −60 mmHg). Negative pressure that is “more” or “greater” than-X mmHg corresponds to pressure that is further from atmospheric pressure (for example, −80 mmHg is more than −60 mmHg). In some cases, local ambient atmospheric pressure is used as a reference point, and such local atmospheric pressure may not necessarily be, for example, 760 mmHg.

Systems and methods disclosed herein can be used with other types of treatment in addition to or instead of reduced pressure therapy, such as irrigation, ultrasound, heat or cold, neuro stimulation, or the like. In some cases, disclosed systems and methods can be used for wound monitoring without application of additional therapy. Systems and methods disclosed herein can be used in conjunction with a dressing, including with compression dressing, reduced pressure dressing, or the like.

A healthcare provider, such as a clinician, nurse, or the like, can provide a TNP prescription specifying, for example, the pressure level or time of application. However, the healing process is different for each patient and the prescription may affect the healing process in a way the clinician or healthcare provider did not expect at the time of devising the prescription. A healthcare provider may try to adjust the prescription as the wound heals (or does not heal), but such process may require various appointments that can be time consuming and repetitive. Embodiments disclosed herein provide systems, devices, or methods of efficiently adjusting TNP prescriptions and delivering effective TNP therapy.

### Wound Therapy System

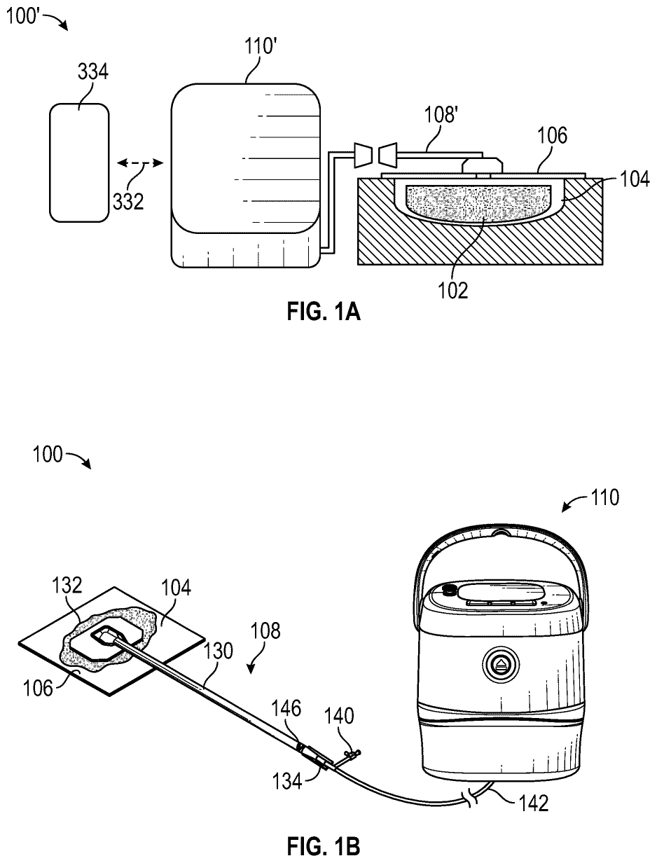

FIG. 1A schematically illustrates a negative pressure wound treatment system 100′ (sometimes referred to as a reduced or negative pressure wound therapy system, a TNP system, or a wound treatment system). In any implementations disclosed herein, though not required, the negative pressure wound treatment system 100′ can include a wound filler 102 placed on or inside a wound 104 (which may be a cavity). The wound 104 can be sealed by a wound cover 106, which can be a drape, such that the wound cover 106 can be in fluidic communication with the wound 104. The wound filler 102 in combination with the wound cover 106 can be referred to as a wound dressing. A tube or conduit 108′ (also referred to herein as a flexible suction adapter or a fluidic connector) can be used to connect the wound cover 106 with a wound therapy device 110′ (sometimes as a whole or partially referred to as a “pump assembly”) configured to supply reduced or negative pressure. The conduit 108′′can be a single or multi lumen tube. A connector can be used to removably and selectively couple a conduit or tube of the device 110′ with the conduit 108′.

In any of the systems disclosed herein, a wound therapy device can be canisterless, wherein, for example and without limitation, wound exudate is collected in the wound dressing or is transferred via a conduit for collection at another location. However, any of the wound therapy devices disclosed herein can include or support a canister.

Additionally, with any of the wound therapy systems disclosed herein, any of the wound therapy devices can be mounted to or supported by the wound dressing or adjacent to the wound dressing. The wound filler 102 can be any suitable type, such as hydrophilic or hydrophobic foam, gauze, inflatable bag, and so on. The wound filler 102 can be conformable to the wound 104 such that the wound filler 102 substantially fills the cavity of the wound 104. The wound cover 106 can provide a substantially fluid impermeable seal over the wound 104. The wound cover 106 can have a top side and a bottom side. The bottom side can adhesively (or in any other suitable manner) seal with the wound 104, for example by sealing with the skin around the wound 104. The conduit 108′ or any other conduit disclosed herein can be formed from polyurethane, PVC, nylon, polyethylene, silicone, or any other suitable material.

The wound cover 106 can have a port (not shown) configured to receive an end of the conduit 108′. In some cases, the conduit 108′ can otherwise pass through or under the wound cover 106 to supply reduced pressure to the wound 104 so as to maintain a desired level of reduced pressure in the wound 104. The conduit 108′ can be any suitable article configured to provide at least a substantially sealed fluid flow pathway or path between the wound therapy device 110′ and the wound cover 106, so as to supply the reduced pressure provided by the wound therapy device 110′ to wound 104.

The wound cover 106 and the wound filler 102 can be provided as a single article or an integrated single unit. In some cases, no wound filler is provided and the wound cover by itself may be considered the wound dressing. The wound dressing can then be connected, via the conduit 108′, to a source of negative pressure of the wound therapy device 110′. In some cases, though not required, the wound therapy device 110′ can be miniaturized and portable, although larger conventional negative pressure sources (or pumps) can also be used.

The wound cover 106 can be located over a wound site to be treated. The wound cover 106 can form a substantially sealed cavity or enclosure over the wound. The wound cover 106 can have a film having a high water vapour permeability to enable the evaporation of surplus fluid, and can have a superabsorbing material contained therein to safely absorb wound exudate. In some cases, the components of the TNP systems described herein can be particularly suited for incisional wounds that exude a small amount of wound exudate.

The wound therapy device 110′ can operate with or without the use of an exudate canister. In some cases, as is illustrated, the wound therapy device 110′ can include an exudate canister. In some cases, configuring the wound therapy device 110′ and conduit 108′ so that the conduit 108′ can be quickly and easily removed from the wound therapy device 110′ can facilitate or improve the process of wound dressing or pump changes, if necessary. Any of the pump assemblies disclosed herein can have any suitable connection between the conduit 108′ and the pump.

The wound therapy device 110′ can deliver negative pressure of approximately −80 mmHg, or between about −20 mmHg and −200 mmHg. Note that these pressures are relative to normal ambient atmospheric pressure thus, −200 mmHg would be about 560 mmHg in practical terms. In some cases, the pressure range can be between about −40 mmHg and −150 mmHg. Alternatively, a pressure range of up to −75 mmHg, up to −80 mmHg or over −80 mmHg can be used. Also in some cases a pressure range of below −75 mmHg can be used. Alternatively, a pressure range of over approximately −100 mmHg, or even −150 mmHg, can be supplied by the wound therapy device 110′.

As will be described in greater detail below, the negative pressure wound treatment system 100′ can be configured to provide a connection 332 to a separate or remote computing device 334. The connection 332 can be wired or wireless (such as, Bluetooth, Bluetooth low energy (BLE). Near-Field Communication (NFC), WiFi, or cellular). The remote computing device 334 can be a smartphone, a tablet, a laptop or another standalone computer, a server (such as, a cloud server), another pump device, or the like.

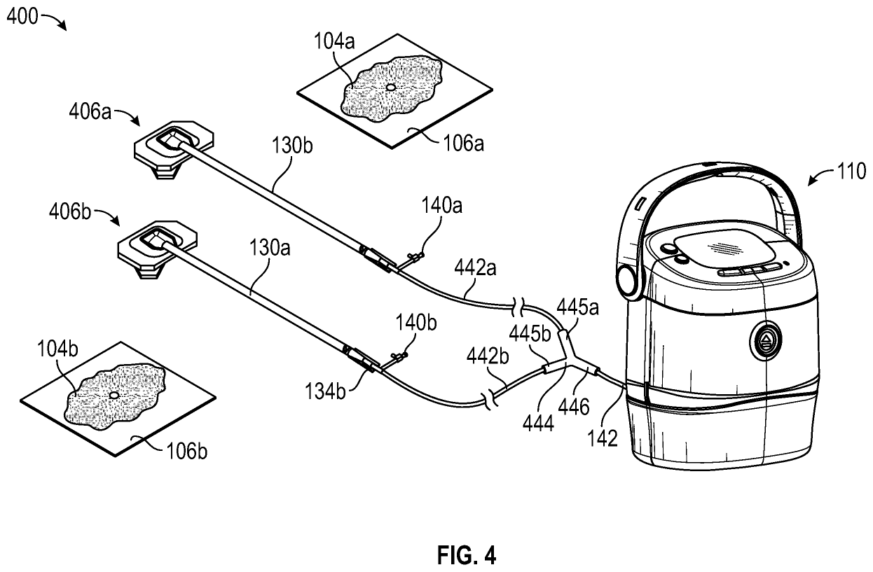

FIG. 1B illustrates another negative pressure wound treatment system 100. The negative pressure wound treatment system 100 can have any of the components, features, or other details of any of the other negative pressure wound treatment system disclosed herein, including without limitation the negative pressure wound treatment system 100′ illustrated in FIG. 1A or the negative pressure wound treatment system 400 illustrated in FIG. 4, in combination with or in place of any of the components, features, or other details of the negative pressure wound treatment system 100 shown in FIG. 1B and/or described herein. The negative pressure wound treatment system 100 can have a wound cover 106 over a wound 104 that can seal the wound 104. A conduit 108, such as a single or multi lumen tube can be used to connect the wound cover 106 with a wound therapy device 110 (sometimes as a whole or partially referred to as a “pump assembly”) configured to supply reduced or negative pressure. The wound cover 106 can be in fluidic communication with the wound 104.

With reference to FIG. 1B, the conduit 108 can have a bridge portion 130 that can have a proximal end portion and a distal end portion (the distal end portion being closer to the wound 104 than the proximal end portion, and an applicator 132 at the distal end of the bridge portion 130 forming the flexible suction adapter (or conduit) 108. A connector 134 can be disposed at the proximal end of the bridge portion 130, so as to connect to at least one of the channels that can extend along a length of the bridge portion 130 of the conduit 108 shown in FIG. 1B. A cap 140 can be coupled with a portion of the conduit 108 and can, in some cases, as illustrated, be attached to the connector 134. The cap 140 can be useful in preventing fluids from leaking out of the proximal end of the bridge portion 130. The conduit 108 can be a Soft Port manufactured by Smith & Nephew. As mentioned, the negative pressure wound treatment system 100 can include a source of negative pressure, such as the device 110, capable of supplying negative pressure to the wound 104 through the conduit 108. Though not required, the device 110 can also include a canister or other container for the storage of wound exudates and other fluids that can be removed from the wound.

The device 110 can be connected to the connector 134 via a conduit or tube 142. In use, the applicator 132 can be placed over an aperture formed in a cover 106 that is placed over a suitably-prepared wound or wound 104. Subsequently, with the wound therapy device 110 connected via the tube 142 to the connector 134, the wound therapy device 110 can be activated to supply negative pressure to the wound. Application of negative pressure can be applied until a desired level of healing of the wound is achieved.

The bridge portion 130 can comprise an upper channel material or layer positioned between an upper layer and an intermediate layer, with a lower channel material or layer positioned between the intermediate layer and a bottom layer. The upper, intermediate, and lower layers can have elongate portions extending between proximal and distal ends and can include a material that is fluid-impermeable, for example polymers such as polyurethane. It will of course be appreciated that the upper, intermediate, and lower layers can each be constructed from different materials, including semi-permeable materials. In some cases, one or more of the upper, intermediate, and lower layers can be at least partially transparent. In some instances, the upper and lower layers can be curved, rounded or outwardly convex over a majority of their lengths.

The upper and lower channel layers can be elongate layers extending from the proximal end to the distal end of the bridge 130 and can each preferably comprise a porous material, including for example open-celled foams such as polyethylene or polyurethane. In some cases, one or more of the upper and lower channel layers can be comprised of a fabric, for example a knitted or woven spacer fabric (such as a knitted polyester 3D fabric, Baltex 7970®, or Gehring 879®) or a nonwoven material, or terry-woven or loop-pile materials. The fibers may not necessarily be woven, and can include felted and flocked (including materials such as Flotex. RTM.) fibrous materials. The materials selected are preferably suited to channeling wound exudate away from the wound and for transmitting negative pressure or vented air to the wound site, and can also confer a degree of kinking or occlusion resistance to the channel layers. In one example, the upper channel layer can include an open-celled foam such as polyurethane, and the lower channel layer can include a fabric. In another example, the upper channel layer is optional, and the system can instead be provided with an open upper channel. The upper channel layer can have a curved, rounded or upwardly convex upper surface and a substantially flat lower surface, and the lower channel layer can have a curved, rounded or downwardly convex lower surface and a substantially flat upper surface.

The fabric or material of any components of the bridge 130 can have a three-dimensional (3D) structure, where one or more types of fibers form a structure where the fibers extend in all three dimensions. Such a fabric can in some cases aid in wicking, transporting fluid or transmitting negative pressure. In some cases, the fabric or materials of the channels can include several layers of material stacked or layered over each other, which can in some cases be useful in preventing the channel from collapsing under the application of negative pressure. The materials used in some implementations of the conduit 108 can be conformable and pliable, which can, in some cases, help to avoid pressure ulcers and other complications which can result from a wound treatment system being pressed against the skin of a patient.

The distal ends of the upper, intermediate, and lower layers and the channel layers can be enlarged at their distal ends (to be placed over a wound site), and can form a “teardrop” or other enlarged shape. The distal ends of at least the upper, intermediate, and lower layers and the channel layers can also be provided with at least one through aperture. This aperture can be useful not only for the drainage of wound exudate and for applying negative pressure to the wound, but also during manufacturing of the device, as these apertures can be used to align these respective layers appropriately.

In some implementations, a controlled gas leak 146 (sometimes referred to as gas leak, air leak, or controlled air leak) can be disposed on the bridge portion 130, for example at the proximal end thereof. This air leak 146 can comprise an opening or channel extending through the upper layer of the bridge portion 130, such that the air leak 146 is in fluidic communication with the upper channel of the bridge portion 130. Upon the application of suction to the conduit 108, gas (such, as air) can enter through the gas leak 146 and move from the proximal end of the bridge portion 130 to the distal end of the bridge portion along the upper channel of the bridge portion 130. The gas can then be suctioned into the lower channel of the bridge portion 130 by passing through the apertures through the distal ends of the upper, intermediate, and lower layers.

The air leak 146 can include a filter. Preferably, the air leak 146 is located at the proximal end of the bridge portion 130 so as to minimize the likelihood of wound exudate or other fluids coming into contact and possibly occluding or interfering with the air leak 146 or the filter. In some instances, the filter can be a microporous membrane capable of excluding microorganisms and bacteria, and which may be able to filter out particles larger than 45 μm. Preferably, the filter can exclude particles larger than 1.0 μm, and more preferably, particles larger than 0.2 μm. Advantageously, some implementations can provide for a filter that is at least partially chemically-resistant, for example to water, common household liquids such as shampoos, and other surfactants. In some cases, reapplication of vacuum to the suction adapter or wiping of the exposed outer portion of the filter may be sufficient to clear any foreign substance occluding the filter. The filter can be composed of a suitably-resistant polymer such as acrylic, polyethersulfone, or polytetrafluoroethylene, and can be oleophobic or hydrophobic. In some cases, the gas leak 146 can supply a relatively constant gas flow that does not appreciably increase as additional negative pressure is applied to the conduit 108. In instances of the negative pressure wound treatment system 100 where the gas flow through the gas leak 146 increases as additional negative pressure is applied, preferably this increased gas flow will be minimized and not increase in proportion to the negative pressure applied thereto. Further description of such bridges, conduits, air leaks, and other components, features, and details that can be used with any implementations of the negative pressure wound treatment systems disclosed herein are found in U.S. Pat. No. 8,801,685, which is incorporated by reference in its entirety as if fully set forth herein.

Any of the wound therapy devices (such as, the device 110 or 110′) disclosed herein can provide continuous or intermittent negative pressure therapy. Continuous therapy can be delivered at above 0 mmHg, −25 mmHg, −40 mmHg, −50 mmHg, −60 mmHg, −70 mmHg, −80 mmHg, −90 mmHg, −100 mmHg, −120 mmHg, −125 mmHg, −140 mmHg, −160 mmHg, −180 mmHg, −200 mmHg, or below-200 mmHg. Intermittent therapy can be delivered between low and high negative pressure set points (sometimes referred to as setpoint). Low set point can be set at above 0 mmHg, −25 mmHg, −40 mmHg, −50 mmHg, −60 mmHg, −70 mmHg, −80 mmHg, −90 mmHg, −100 mmHg, −120 mmHg, −125 mmHg, −140 mmHg, −160 mmHg, −180 mmHg, or below −180 mmHg. High set point can be set at above −25 mmHg, −40 mmHg, −50 mmHg, −60 mmHg, −70 mmHg, −80 mmHg, −90 mmHg, −100 mmHg, −120 mmHg, −125 mmHg, −140 mmHg, −160 mmHg, −180 mmHg, −200 mmHg, or below −200 mmHg. During intermittent therapy, negative pressure at low set point can be delivered for a first time duration, and upon expiration of the first time duration, negative pressure at high set point can be delivered for a second time duration. Upon expiration of the second time duration, negative pressure at low set point can be delivered. The first and second time durations can be same or different values.

In operation, the wound filler 102 can be inserted into the cavity of the wound 104, and wound cover 106 can be placed so as to seal the wound 104. The wound therapy device 110′ can provide negative pressure to the wound cover 106, which can be transmitted to the wound 104 via the wound filler 102. Fluid (such as, wound exudate) can be drawn through the conduit 108′ and stored in a canister. In some cases, fluid is absorbed by the wound filler 102 or one or more absorbent layers (not shown).

Wound dressings that can be utilized with the pump assembly and systems of the present application include Renasys-F, Renasys-G, Renasys AB, and Pico Dressings available from Smith & Nephew. Further description of such wound dressings and other components of a negative pressure wound therapy system that can be used with the pump assembly and systems of the present application are found in U.S. Patent Publication Nos. 2012/0116334, 2011/0213287, 2011/0282309, 2012/0136325, U.S. Pat. No. 9,084,845, and International App. No. PCT/EP2020/078376, each of which is incorporated by reference in its entirety as if fully set forth herein. In some cases, other suitable wound dressings can be utilized.

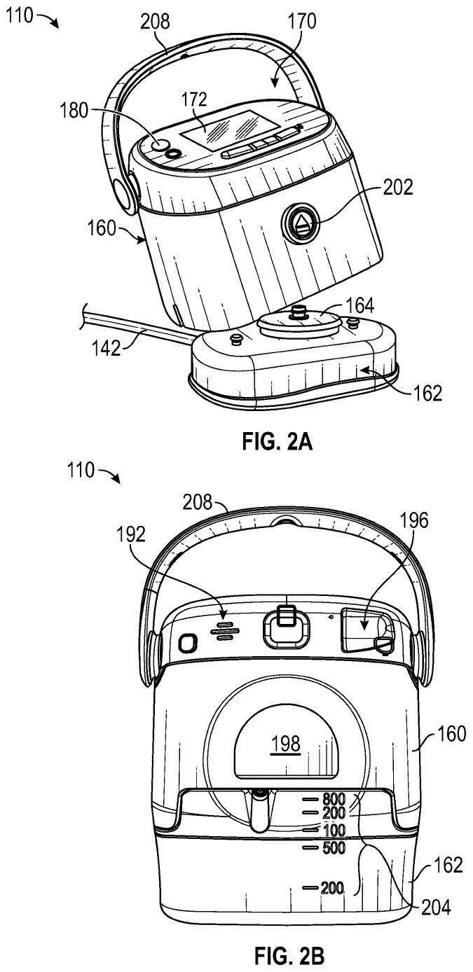

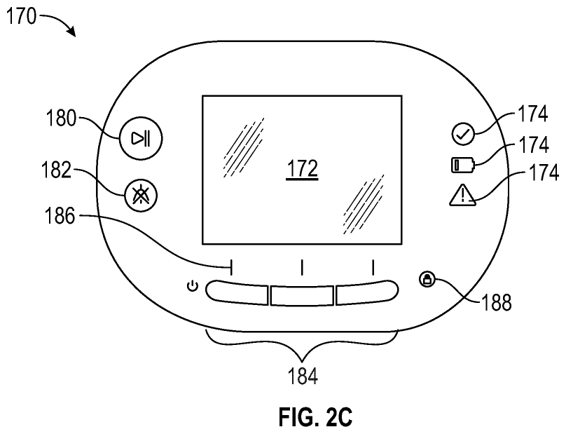

FIGS. 2A-2C show the negative pressure wound therapy device 110. As illustrated, a pump assembly 160 and canister 162 can be connected, thereby forming the wound therapy device 110. With reference to FIG. 2C, the pump assembly 160 can include an interface panel 170 having a display 172, one or more indicators 174, or one or more controls or buttons, including, for example and without limitation, a therapy start and pause button 180 or an alarm/alert mute button 182. The interface panel 170 can have one or more input controls or buttons 184 (three being shown) that can be used to control any functions of the pump assembly 160 or the interface panel 170. For example and without limitation, one or more of the buttons 184 can be used to turn the pump assembly 160 on or off, to start or pause therapy, to operate and monitor the operation of the pump assembly 160, to scroll through menus displayed on the display 172, or to control or perform other functions. In some cases, the command buttons 184 can be programmable, and can be made from a tactile, soft rubber.

Additionally, the interface panel 170 can have visual indicators 186 that can indicate which of the one or more buttons 184 is active. The interface panel 170 can also have a lock/unlock control or button 188 that can be configured to selectively lock or unlock the functionality of the various buttons (e.g., buttons 184) or the display 172. For example, therapy setting adjustment can be locked/unlocked via the lock/unlock control 188. When the lock/unlock button 188 is in the locked state, depressing one or more of the various other buttons or the display will not cause the pump assembly 160 to change any display functions or performance functions of the device. This way, the interface panel 170 will be protected from inadvertent bumping or touching of the various buttons or display. The interface panel 170 can be located on an upper portion of the pump assembly 160, for example and without limitation on an upward facing surface of the pump assembly 160.

The display 172, which can be a screen such as an LCD screen, can be mounted in a middle portion of the interface panel 170. The display 172 can be a touch screen display. The display 172 can support playback of audiovisual (AV) content, such as instructional videos, and render a number of screens or graphical user interfaces (GUIs) for configuring, controlling, and monitoring the operation of the pump assembly 160.

The one or more indicators 174 can be lights (such as, LEDs) and can be configured to provide a visual indication of alarm conditions and or a status of the pump. For example and without limitation, the one or more indicators 174 can be configured to provide a visual indication of a status of the pump assembly 160 or other components of the negative pressure wound treatment system 100, including without limitation the conduit 108 or the wound cover 106 (such as, to provide an indication of normal operation, low battery, a leak, canister full, blockage, overpressure, or the like). Any one or more suitable indicators can be additionally or alternatively used, such as visual, audio, tactile indicator, and so on.

FIG. 2B shows a back or rear view of the wound therapy device 110 shown in the FIG. 2A. As shown, the pump assembly 160 can include a speaker 192 for producing sound. For example and without limitation, the speaker 192 can generate an acoustic alarm in response to deviations in therapy delivery, non-compliance with therapy delivery, or any other similar or suitable conditions or combinations thereof. The speaker 192 can provide audio to accompany one or more instructional videos that can be displayed on the display 172.

The pump assembly 160 can be configured to provide easy access (such as, an access door on the casing of the pump assembly) to one or more filters of the pump assembly 160, such as antibacterial filters. This can enable a user (such as, a healthcare provider or patient) to more easily access, inspect or replace such filters. The pump assembly 160 can also include a power jack (or socket) 196 for providing power to the pump assembly 160 or for charging and recharging an internal power source (such as, a battery). Some implementations of the pump assembly 160 can include a disposable or renewable power source, such as one or more batteries, so that no power jack is needed. The pump assembly 160 can have a recess 198 formed therein to facilitate gripping of the pump assembly 160.

The canister 162 can hold fluid aspirated from the wound 104. For example, the canister 162 can have an 800 mL (or approximately 800 mL) capacity, or from a 300 mL or less capacity to a 1000 mL or more capacity, or any capacity level in this range. The canister 162 can include a tubing for connecting to the conduit 108 in order to form a fluid flow path. The canister 162 can be replaced with another canister, such as when the canister 162 has been filled with fluid. With reference to FIG. 2A, the wound therapy device 110 can include a canister inlet tube 142 (also referred to herein as a dressing port connector) in fluid communication with the canister 162. For example and without limitation, the canister inlet tube 142 can be used to connect with the conduit 108.

The canister 162 can be selectively coupleable and removable from the pump assembly 160. With reference to FIG. 2A, in some cases, a canister release button 202 can be configured to selectively release the canister 162 from the pump assembly 160. With reference to FIG. 2B, the canister 162 can have one or more fill lines or graduations 204 to indicate to the user and amount of fluid or exudate stored within the canister 162.

The wound therapy device 110 can have a handle 208 that can be used to lift or carry the wound therapy device 110. The handle 208 can be coupled with the pump assembly 160 and can be rotatable relative to the wound therapy device 110 so that the handle can be rotated upward for lifting or carrying the wound therapy device 110 or the pump assembly 160, or rotated into a lower profile in a more compact position when the handle is not being used. In some cases, the handle 208 can be coupled with the pump assembly 160 in a fixed position. The handle 208 can be coupled with an upper portion of the pump assembly 160 or can be removable from the wound therapy device 110.

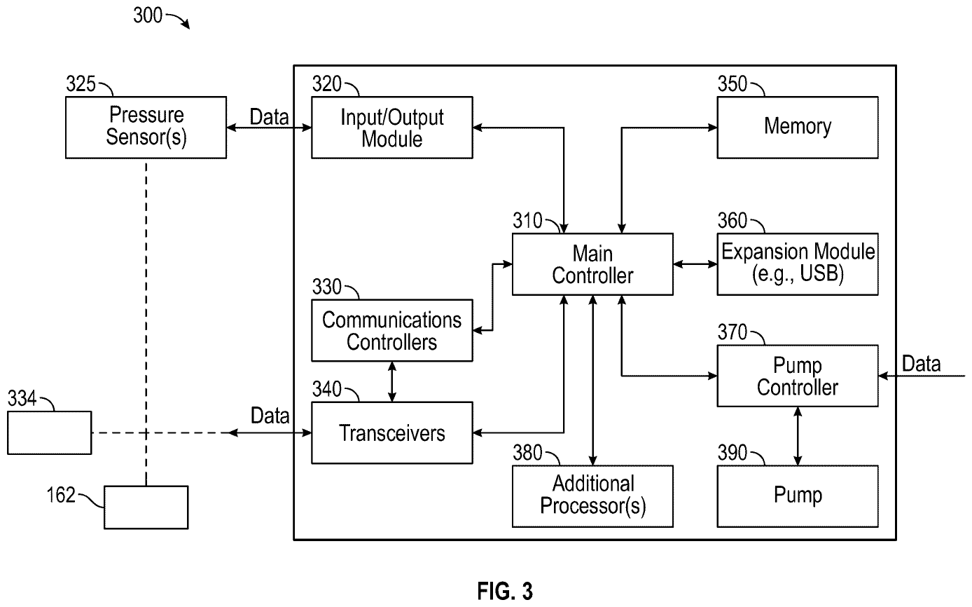

FIG. 3 illustrates a schematic of a control system 300 that can be employed in any of the wound therapy devices described herein, such as in the wound therapy device 110. Electrical components can operate to accept user input, provide output to the user, operate the pressure source, provide connectivity, and so on. A first processor (such as, a main controller 310) can be responsible for user activity, and a second processor (such as, a pump controller 370) can be responsible for controlling another device, such as a pump 390.

An input/output (I/O) module 320 can be used to control an input and/or output to another component or device, such as the pump 390, one or more sensors (for example, one or more pressure sensors 325 configured to monitor pressure in one or more locations of the fluid flow path), or the like. For example, the I/O module can receive data from one or more sensors through one or more ports, such as serial (for example, I2C), parallel, hybrid ports, and the like. Any of the pressure sensors can be part of the wound therapy device or the canister. In some cases, any of the pressure sensors 325 can be remote to the wound therapy device, such as positioned at or near the wound (for example, in the dressing or the conduit connecting the dressing to the wound therapy device). In such implementations, any of the remote pressure sensors can communicate with the I/O module over a wired connection or with one or more transceivers 340 over a wireless connection.

The main controller 310 can receive data from and provide data to one or more expansion modules 360, such as one or more USB ports, SD ports, Compact Disc (CD) drives, DVD drives, FireWire ports, Thunderbolt ports, PCI Express ports, and the like. The main controller 310, along with other controllers or processors, can store data in memory 350 (such as one or more memory modules), which can be internal or external to the main controller 310. Any suitable type of memory can be used, including volatile or non-volatile memory, such as RAM, ROM, magnetic memory, solid-state memory, Magnetoresistive random-access memory (MRAM), and the like.

The main controller 310 can be a general purpose controller, such as a low-power processor or an application specific processor. The main controller 310 can be configured as a “central” processor in the electronic architecture of the control system 300, and the main controller 310 can coordinate the activity of other processors, such as the pump controller 370, one or more communications controllers 330, and one or more additional processors 380. The main controller 310 can run a suitable operating system, such as a Linux, Windows CE, VxWorks, etc.

The pump controller 370 can control the operation of a pump 390, which can generate negative or reduced pressure. The pump 390 can be a suitable pump, such as a diaphragm pump, peristaltic pump, rotary pump, rotary vane pump, scroll pump, screw pump, liquid ring pump, diaphragm pump operated by a piezoelectric transducer, voice coil pump, and the like. The pump controller 370 can measure pressure in a fluid flow path, using data received from one or more pressure sensors 325, calculate the rate of fluid flow, and control the pump. The pump controller 370 can control the pump actuator (such as, a motor) so that a desired level of negative pressure is achieved in the wound 104. The desired level of negative pressure can be pressure set or selected by the user. The pump controller 370 can control the pump (for example, pump motor) using pulse-width modulation (PWM) or pulsed control. A control signal for driving the pump can be a 0-100% duty cycle PWM signal. The pump controller 370 can perform flow rate calculations and detect alarms. The pump controller 370 can communicate information to the main controller 310. The pump controller 370 can be a low-power processor.

Any of the one or more communications controllers 330 can provide connectivity (such as, a wired or wireless connection 332). The one or more communications controllers 330 can utilize one or more transceivers 340 for sending and receiving data. The one or more transceivers 340 can include one or more antennas, optical sensors, optical transmitters, vibration motors or transducers, vibration sensors, acoustic sensors, ultrasound sensors, or the like. Any of the one or more transceivers 340 can function as a communications controller. In such case, the one or more communications controllers 330 can be omitted. Any of the one or more transceivers 340 can be connected to one or more antennas that facilitate wireless communication. The one or more communications controllers 330 can provide one or more of the following types of connections: Global Positioning System (GPS), cellular connectivity (for example, 2G, 3G, LTE, 4G, 5G, or the like), NFC, Bluetooth (or BLE) connectivity, radio frequency identification (RFID), wireless local area network (WLAN), wireless personal area network (WPAN), WiFi connectivity, Internet connectivity, optical connectivity (for example, using infrared light, barcodes, such as QR codes, etc.), acoustic connectivity, ultrasound connectivity, or the like. Connectivity can be used for various activities, such as pump assembly location tracking, asset tracking, compliance monitoring, remote selection, uploading of logs, alarms, and other operational data, and adjustment of therapy settings, upgrading of software or firmware, pairing, and the like.

Any of the one or more communications controllers 330 can provide dual GPS/cellular functionality. Cellular functionality can, for example, be 3G, 4G, or 5G functionality. The one or more communications controllers 330 can communicate information to the main controller 310. Any of the one or more communications controllers 330 can include internal memory or can utilize memory 350. Any of the one or more communications controllers 330 can be a low-power processor.

The control system 300 can store data, such as GPS data, therapy data, device data, and event data. This data can be stored, for example, in memory 350. This data can include patient data collected by one or more sensors. The control system 300 can track and log therapy and other operational data. Such data can be stored, for example, in the memory 350.

Using the connectivity provided by the one or more communications controllers 330, the control system 300 can upload any of the data stored, maintained, or tracked by the control system 300 to a remote computing device, such as the device 334. The control system 300 can also download various operational data, such as therapy selection and parameters, firmware and software patches and upgrades, and the like (for example, via the connection to the device 334). The one or more additional processors 380, such as processor for controlling one or more user interfaces (such as, one or more displays), can be utilized. In some cases, any of the illustrated or described components of the control system 300 can be omitted depending on an embodiment of a wound monitoring or treatment system in which the control system 300 is used.

Using connectivity provided by the one or more communications controllers 330, the control system 330 can communicate with a canister, such as the canister 162. The canister 162 can report operating status to the control system 330, such as, whether the canister is full, level of fluid stored in the canister, or the like.

Any of the negative pressure wound therapy devices described herein can include one or more features disclosed in U.S. Pat. No. 9,737,649 or U.S. Patent Publication No. 2017/0216501, each of which is incorporated by reference in its entirety.

### Multiple Dressing Negative Wound Therapy

FIG. 4 illustrates another negative pressure wound treatment system 400. The system 400 can include a wound therapy device capable of supplying negative pressure to the wound site or sites, such as wound therapy device 110. The wound therapy device 110 can be in fluidic communication with one or more wound dressings 406a, 406b (collectively referred to as 406) so as to supply negative pressure to one or more wounds, such as the wounds 104 a and 104b. A first fluid flow path can include components providing fluidic connection from the wound therapy device 110 to the first wound dressing 406a. As a non-limiting example, the first fluid flow path can include the path from the wound dressing 406a to the wound therapy device 110 or the path from the first wound dressing 406a to an inlet 446 of a branching attachment (or connector) 444 in fluidic connection with the wound therapy device 110. Similarly, a second fluid flow path can include components providing fluidic connection from the wound therapy device 110 to the second wound dressing 406b.

The system 400 can be similar to the system 100 with the exception that multiple wounds 104a and 140b are being treated by the system 400. The system 400 can include any one or more of the components of the system 100, which are illustrated in FIG. 4 with appended letter “a” or “b” to distinguish between the first and second wounds (such as, the wounds 104a and 104b, the covers 106a and 106b). As illustrated, the system 400 can include a plurality of wound dressings 406a, 406b (and corresponding fluid flow paths) in fluidic communication with the wound therapy device 110 via a plurality of suction adapters, such as the adapter 108. The suction adapters can include any one or more of the components of the adapter 108, which are illustrated in FIG. 4 with appended letter “a” or “b” to distinguish between the first and second wounds (such as, the bridge portions 130a and 130b, the connectors 134a and 134b, and the caps 140a and 140b).

The wound therapy device 110 can be fluidically coupled via the tube 142 with the inlet 446 of the connector 444. The connector 444 can be fluidically coupled via branches 445a, 445b and tubes or conduits 442a, 442b with the connectors 134a, 134b, which can be fluidically coupled with the tubes or conduits 130a, 130b. The tubes or conduits 130a, 130b can be fluidically coupled with the dressings 406a, 406b. Once all conduits and dressing components are coupled and operably positioned, the wound therapy device 110 can be activated, thereby supplying negative pressure via the fluid flow paths to the wounds 430a, 430b. Application of negative pressure can be applied until a desired level of healing of the wounds 430 is achieved. Although two wounds and wound dressing are illustrated in FIG. 4, some implementations of the wound therapy device 110 can provide treatment to a single wound (for instance, by closing the unused branch 445a or 445b of the connector 444) or to more than two wounds (for instance, by adding branches to the connector 444).

The system 400 can include one or more features disclosed in U.S. Patent Publication No. 2020/0069850 or International Publication No. WO 2018/167199, each of which is incorporated by reference in its entirety.

### Multiple Communications Modes

A negative pressure wound therapy device, such as the pump assembly 160, can communicate with multiple devices or components. The communicated data can include operational data related to the provision of TNP therapy. Communication of data by the pump assembly 16 can, for instance, facilitate remote monitoring and troubleshooting, which can improve effectiveness of TNP therapy.

For example, as described herein, the pump assembly 160 can communicate with the remote computing device 334 over the connection 332. The connection 332 can be a wireless connection, such as Bluetooth (or BLE), WiFi, etc. The pump assembly 160 can communicate with the canister 162, as described herein. Same or different communication protocols can be implemented by the pump assembly 160 for communicating with multiple devices or negative pressure wound therapy system components.

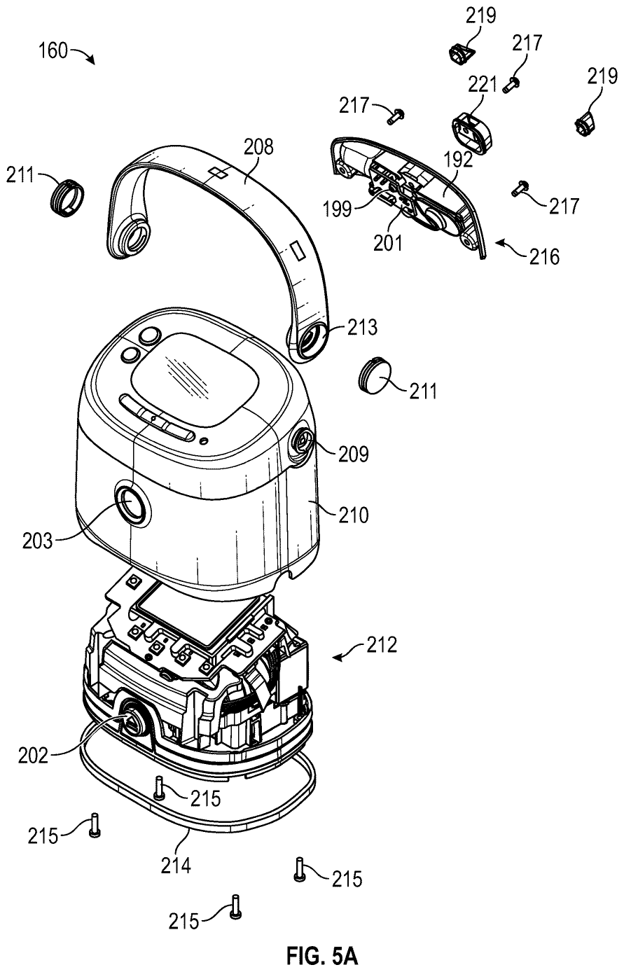

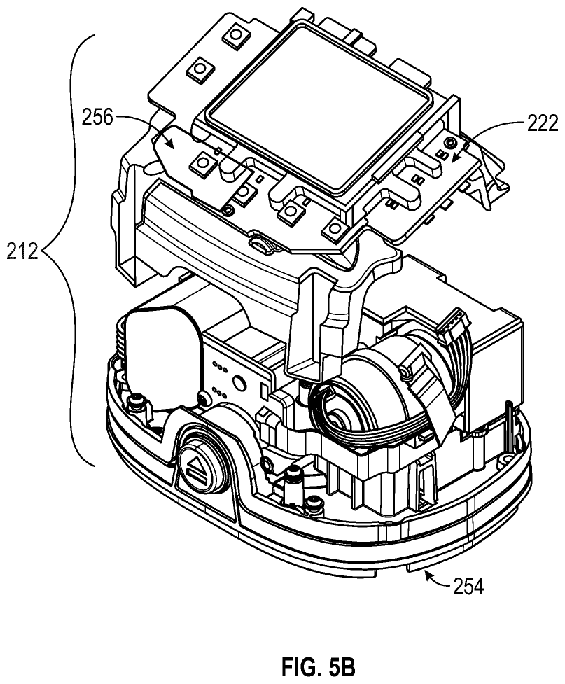

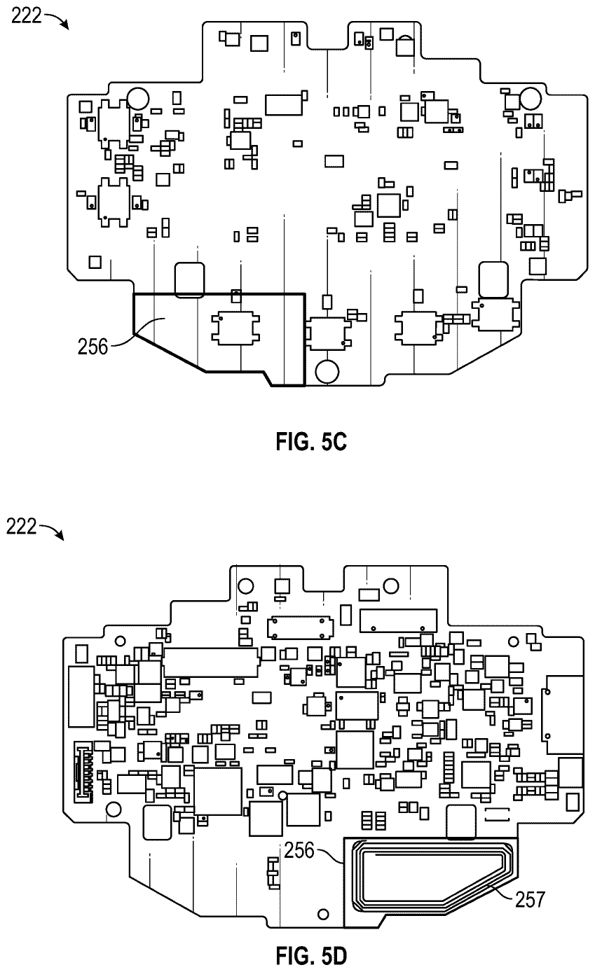

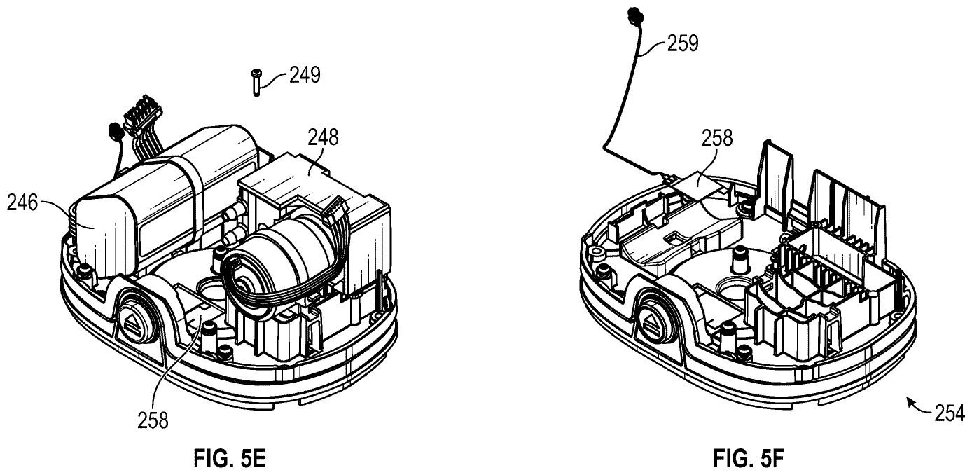

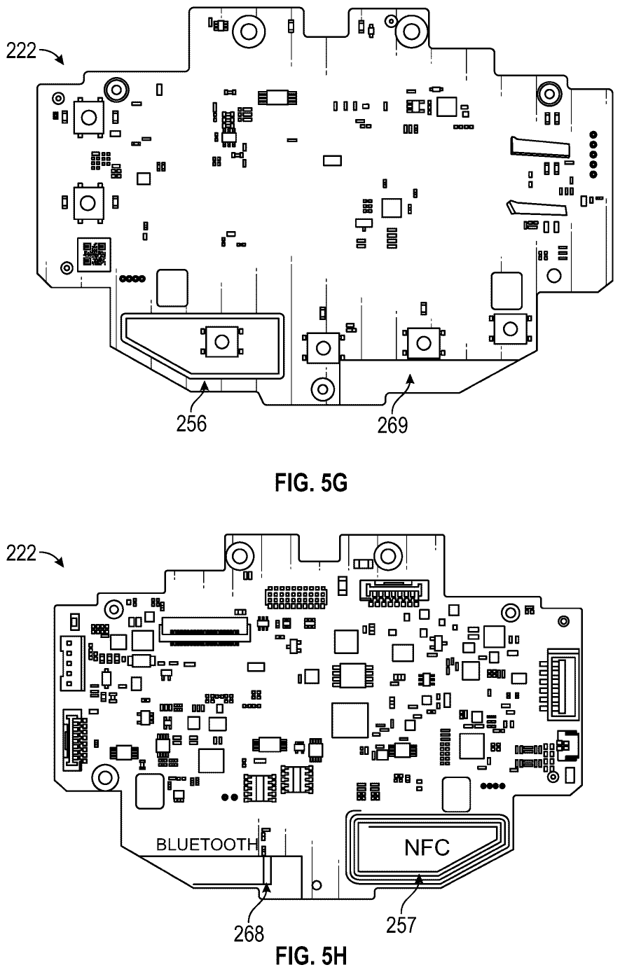

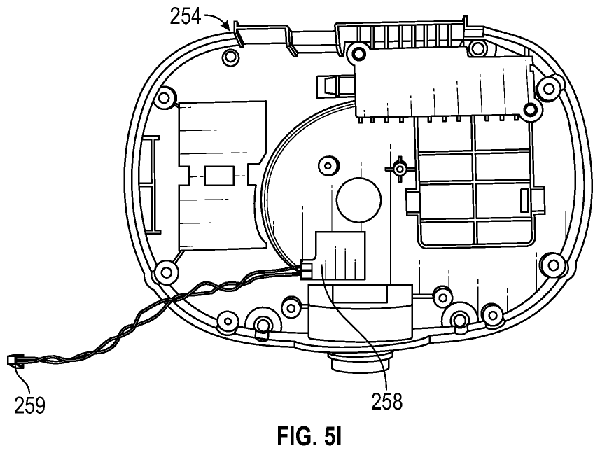

FIG. 5A illustrates an exploded view of the pump assembly 160. Some arrangements of the pump assembly 160 have been designed to facilitate the ease with which components and sub-components of the pump assembly 160 can be removed from the pump assembly 160 for cleaning, servicing, replacing, or otherwise. Some arrangements of the pump assembly 160 are designed such that the various components and/or sub-assemblies are arranged and assembled in the pump assembly 160 in a modular fashion to make the various components and sub-assemblies easier to remove, clean, and/or service. In some arrangements, the pump assembly 160 can have a housing 210 which is sized and configured to enclose at least a core assembly 212. The housing 210 can be made from acrylonitrile butadiene styrene, or any other desirable or suitable materials.

A core base seal 214 can be used to seal the core assembly 212 to the housing 210 of the pump assembly 160. A plurality of screws or other fasteners 215 can be used to couple a core assembly 212 with the housing 210. A rear trim assembly 216 can be coupled with the housing 210 using one or more fasteners 217 that can have one or more screw or fastener covers 219. Some arrangements of the rear trim assembly 216 can have a USB port 199 or other wired connection ports coupled with a printed circuit board (PCB) 201 that can be included in the rear trim assembly 216. The rear trim assembly 216 can include the speaker 192 and can have other components or connectors, buttons, switches, or inputs. A USB cover 221 can be removably coupled with the USB port. A power cord (not shown) can be removably connected to the power jack 196 and extend through the rear trim assembly 216 and be electrically coupled with electrical components of the rear trim assembly 216. Connecting the power cord to the rear trim assembly 216 through the power jack 196 can, in some arrangements, make it easier to remove and replace a damaged power cord as compared to models in which the power cord is connected directly to the internal electronic components within the pump, which can require that the service technician disassemble the pump to replace the power cord. Such arrangement can make it easier to remove and replace a damaged power jack 196. In some arrangements of the pump assembly 160, the service technician need only remove and replace the rear trim assembly 216 to replace the power jack 196.

The handle 208 can be coupled with posts 209 of the housing 210 configured to pivot relative to the housing 210. In some arrangements, the handle can be made from a thermoplastic elastomer or any other suitable or desired material. In some arrangements, the handle can be configured to simply clip or snap onto the housing 210. In some arrangements, the handle 208 can be configured to rotate relative to the housing 210, or can be rigidly (nonrotatably) attached to the housing 210. The handle 208 can include one or more detents to facilitate retaining the handle in the upright position. In some arrangements, two or more handle caps 211 can be used to couple the handle 208 with the posts 209 of the housing 210 or can be used to cover the depressions or recesses 213 in the handle 208. The canister release button 202 can extend through an opening 203 in the housing 210.

As described herein, the pump assembly 160 can communicate with the remote computing device 334. The communication can be performed over the connection 332 using a communication protocol, such as Bluetooth (or BLE), WiFi, etc. For security and integrity, including protection of patient medical data, ensuring that correct remote computing device is connected to correct pump assembly (as there may be many remote computing devices in a vicinity of a pump assembly and vice versa), or the like, it can be advantageous to pair or bond a remote computing device with the pump assembly. Pairing can be performed before or as part of establishing the connection to the remote computing device.

In some cases, pairing can be performed using “just works” approach by which a known key (for example, 0 or another suitable value) is exchanged between the pump assembly and the remote computing device. The temporary key can then be used to establish the remote communication, encrypt the remote communication, or the like. While this approach may not require user interaction, it may not be secure and/or facilitate connection between the correct devices, or the like. These shortcoming may be due to the key being known.

In some cases, pairing can be performed using “passkey” approach by which a key is passed between the remote computing device and the pump assembly by the user. For example, a random key can be generated and displayed on the pump assembly (or on the remote computing device). The user can enter the key into the remote computing device (or into the pump assembly). The key can then be used to establish the remote communication, encrypt the remote communication, or the like. While this approach may facilitate security, connection between the correct devices, or the like, it may undesirably require user interaction.

In some cases, pairing can be performed by exchanging pairing data between the remote computing device and the pump assembly device using a communication protocol that is different from that used by the connection 332. For example, a key can be generated and exchanged between the remote computing device and the pump assembly using inductive coupling, NFC (for instance, passive NFC), RFID (for example, passive RFID), optical communication, or the like. As another example, an IP address (and/or credentials) can be exchanged to establish a WiFi connection, which may be used to then establish a Bluetooth (or BLE) connection. This can be performed when the remote computing device is positioned in physical contact with or within a threshold distance of one or more regions of the pump assembly. The key (or IP address) can be randomly generated. The key (or IP address) can be used to establish the remote communication, encrypt the remote communication, or the like. The remote communication can utilize Bluetooth (or BLE), WiFi, or the like. Advantageously, this approach can facilitate security, connection between the correct devices, etc. while not requiring any user interaction or requiring minimal user interaction.

In some cases, the user may be requested to authorize or confirm the pairing. This can be done by pressing a button of the pump assembly 160, such as any of buttons 184, touching the display 172, or the like. Alternatively or additionally, the user can authorize the pairing on the remote computing device. Additional approaches for exchanging pairing data are disclosed in International Patent Application No. PCT/EP2020/078551, which is incorporated by reference in its entirety.

FIG. 5B illustrates the core assembly 212 and a lower core assembly 254. A printed circuit board (PCB) 222, which can be positioned on the top of the core assembly 212, can support a plurality of electronic components, such as one or more of the components of the control system 300 illustrated in FIG. 3. The PCB 222 can be single-sided (such as, include a single layer of conductive material, such as copper), double-sided (such as, include two layers of conductive material on the top and bottom sides), or multi-layered (such as, include more than two layers of conductive material). One or more antennas for facilitating wireless communications can be supported by or connected to the PCB 222 (such as, to a transceiver supported by the PCB). As described herein, an antenna can be positioned on the PCB 222. With reference to FIGS. 5C and 5D (as well as FIGS. 5G and 5H), which illustrate mirrored views of the top side (for instance, supporting the screen) and bottom side of the PCB 222 respectively, an antenna 257 can be positioned in the region 256. As is illustrated, the antenna 257 can be positioned on the bottom side (or the side facing toward the canister) of the PCB 222. The antenna 257 can facilitate NFC communication. As is illustrated, the antenna 257 can be a coil (or loop) antenna.

The region 256 can correspond to a location or area for pairing the pump assembly 160 with a remote computing device, as described herein. For example, pairing data (such as, a key) for communicating with the remote computing device 334 over a Bluetooth (or BLE) connection can be exchanged via an NFC connection facilitated by an antenna located in the region 256. To facilitate convenience and security, the region 256 can be located in a corner of the pump assembly 160, such as, in the bottom left corner. In some cases, the region 256 can be located in another corner or position.

The region 256 can correspond to coverage area(s) of one or more antennas configured to facilitate the pairing. Coverage area of an antenna, such as the antenna 257, can be associated with space in which the antenna is configured to receive and/or transmit radio frequency (RF) waves or signals. Coverage area of the antenna used for pairing can be limited to include the area enclosed by a portion of the housing 210 overlying the region 256 and/or volume extending a threshold distance away from such area. The threshold distance can be a small distance away from the surface of the housing 210, such as 0.5 cm or less, 1 cm or less, 2 cm or less or more, 3 cm or less or more, 4 cm or less or more, 5 cm or less or more, 10 cm or less or more, 15 cm or less or more. Unless a remote computing device is positioned within the coverage area of the antenna (such as, in contact with such area or within such volume), pairing will not be initiated. Limiting the coverage area of the one or more antennas can improve security (for example, by preventing hacking) and promote connection between the correct devices. In some cases, to further promote security, the one or more regions may not be marked by any indicia (such that, the user may need to a priori know the location(s) of the one or more regions to initiate the pairing). Additional approaches for limiting the coverage area are disclosed in International Patent Application No. PCT/EP2020/078551 (published as WO 2021/074052), which is incorporated by reference in its entirety.

With reference to FIGS. 5E and 5F, the core assembly 212 can include a battery module 246 and a pump motor assembly 248. The pump motor assembly 248 can include a pump motor. The pump motor assembly 248 can include an inlet tubing connector and an outlet tubing connector to which tubing can be coupled. Some arrangements of the pump motor assembly 248 can include a pump controller, such as the pump controller 370. The battery module 246 can be any suitable battery pack and can include single use and rechargeable type batteries such as lithium ion batteries. Some arrangements of the battery can include lithium ion 18650 or 21600 cells, or any other type of battery that has plentiful power supply, has good power density for size, and/or is lightweight. The battery module can include a carefully designed charging circuitry with full redundancy due to the inherent risks of lithium ion battery technology, and can be configured to operate across a limited temperature range.

The pump motor assembly 248 can include a pump motor and a controller. The pump can be a diaphragm pump or any other desired or suitable type of pump, including a rotary pump, a peristaltic pump, a piezoelectric pump, or otherwise. Some arrangements of diaphragm pumps are well suited to the flow rates and pressures required, have a long maintenance-free service life, and are relatively efficient and quiet in operation.

The battery module 246 and/or the pump motor assembly 248 can be coupled with or supported by the lower core assembly 254. One or more fasteners 249 and/or cable ties can be used to couple the pump motor assembly 248 with the other components of the core assembly 212.

An antenna 258 can be provided as illustrated in FIGS. 5E, 5F, and 5I. The antenna 258 can be configured to facilitate communication with a canister, such as the canister 162. The pump assembly 160 can receive data relating to the status of the canister 162 (such as whether the canister is full or the level of fluid in the canister), configuration data (such as, canister capacity), identification data (such as, batch code or serial number of the canister), date of manufacture of the canister (which can be a timestamp), date of first use of the canister (which can be a timestamp), or the like. Such data can be individually or collectively referred to as canister data. In some implementations, exchange of canister data between the pump assembly 160 and the canister 162 (such as, using the antenna 258 and a corresponding antenna in the canister) can indicate presence or positioning of the canister. For instance, reading the canister identification data can provide indication of the canister being connected to the pump assembly 160 (such as, to the housing of the pump assembly 160 as illustrated in FIG. 2A). If it is determined that the canister is not connected and provision of negative pressure wound therapy is requested, an indication can be provided that the canister has not been detected. Provision of therapy (such as, activating the negative pressure source) may be disallowed until a canister has been connected and its presence has been detected.

In some cases, the antenna 258 can support communication using NFC, RFID, or the like. The antenna 258 can be a coil or loop antenna. To efficiently communicate with the canister, the antenna 258 can be positioned at the bottom of the core assembly 212. As illustrated in FIGS. 5F and 5I, the antenna 258 can be positioned near the inlet (located in the hole shown in FIGS. 5F and 5I) of the lower core assembly 254. The antenna 258 can be supported by or be adjacent to the lower core assembly 254. The antenna 258 can plug into the PCB 222 via a connector 259. NFC antenna of the canister can be positioned at or near the top portion of the canister, such as in the filter assembly 164 illustrated in FIG. 2A. Additional approaches for communicating the with the canister are disclosed in co-pending Patent Application No. EP 21382346.1 filed on Apr. 21, 2021 (Atty. Docket SMNPH. 672EP), titled “Canister Status Determination for Negative Pressure Wound Therapy Devices,” which is incorporated by reference in its entirety.

As described herein, the antenna 257 can be positioned at or near the top portion of the pump assembly 160 to facilitate communication with a remote computing device, such as the remote computing device 334. The antenna 257 can be used for exchanging pairing data with the remote computing device. In addition to pairing data, a uniform resource locator (URL) or similar information can be transmitted to the remote computing device. This additional information can be used to access a website for obtaining instructions related to the pump assembly 160, downloading an app or the like for communicating with the pump assembly 160, or the like. For instance, the antenna 257 can support a near-field protocol, which may function over a limited range or distance (such as, 4 cm or less). In some instances, the near-field protocol supported by the antenna 257 can include one or more of ISO/IEC 14443, ISO/IEC 15693, ISO/IEC 18092, or ISO/IEC 21481. The antenna 257 can be part of a passive NFC device (or a tag). A passive NFC tag may be configured to only send data. The passive tag can send the pairing data (such as, a key stored in the memory) to the remote computing device positioned in proximity of the tag (such as, within the coverage area of the antenna 257). Subsequent to pairing, communication with the remote computing device can be performed using another communication protocol than that provided by the antenna 257. Another communication protocol that facilitates exchange of data over a longer distance (such as, Bluetooth or BLE) can be utilized subsequent to pairing. The pump assembly 160 can include another antenna (such, as an antenna 268 illustrated in FIG. 5H) to support another communication protocol, such as a Bluetooth or BLE antenna. A Bluetooth or BLE antenna can be supported by the PCB 222 (such, as be a trace on the PCB 222) or positioned within the housing, as described herein. With reference to FIGS. 5G and 5H, the antenna 268 can be positioned on the bottom side of the PCB 222 in the region 269. The antenna 268 can be a planar inverted-F antenna (PIFA) as shown in FIG. 5H.

In some instances, one or more pressure sensors can be positioned in the fluid flow path remotely from the pump assembly 160. For example, one or more pressure sensors can be positioned at or near the wound (for example, in the dressing or the conduit connecting the dressing to the pump assembly 160). Further approaches to positioning the one or more pressure sensors are described in International Patent Application No. PCT/EP2020/072663 and U.S. Patent Publication No. 2021/0001019, each of which is incorporated by reference in its entirety. The one or more pressure sensors can communicate with the pump assembly 160 similarly to the remote computing device 334. For instance, the one or more pressure sensors can communicate with the pump assembly 160 using Bluetooth (or BLE). Pairing of the one or more pressure sensors can be performed similarly to pairing the remote computing device. Alternatively, in some instances, the pump assembly 160 can pair with a pressure sensor by varying provision of negative pressure, as described in International Patent Application No. PCT/EP2020/072663.

As described herein, a further antenna (such as, the antenna 258) can be positioned at or near the bottom portion of the pump assembly 160 to facilitate communication with a canister, such as the canister 162. The antenna 258 can be part of an active NFC device (or tag), which can be configured to both send and receive data. A passive NFC tag can be positioned in the canister 162 and send canister data. In some cases, the antennas 257 and 258 can support the same communication protocol, such as the same near-field protocol. In such case, only one of antennas 257 or 258 may be configured to operate (such as, receive or transmit data) at a given time in order to reduce or remove any interference resulting from both antennas operating simultaneously.

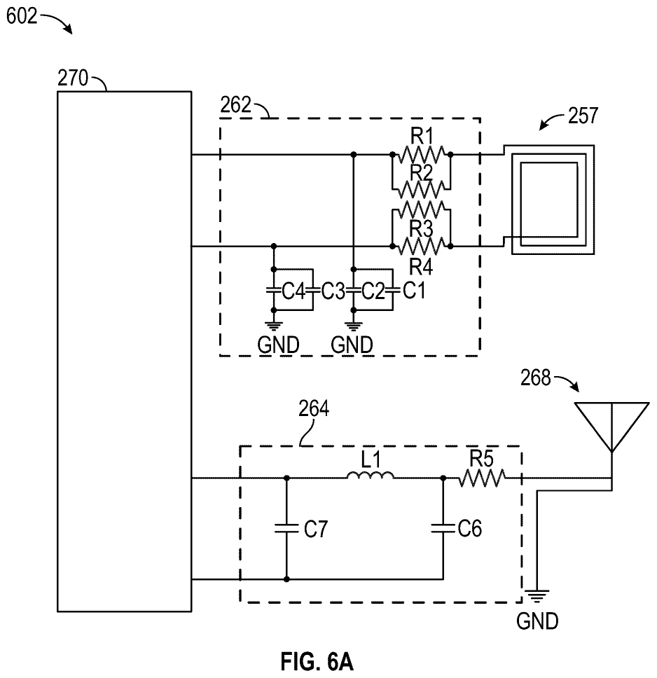

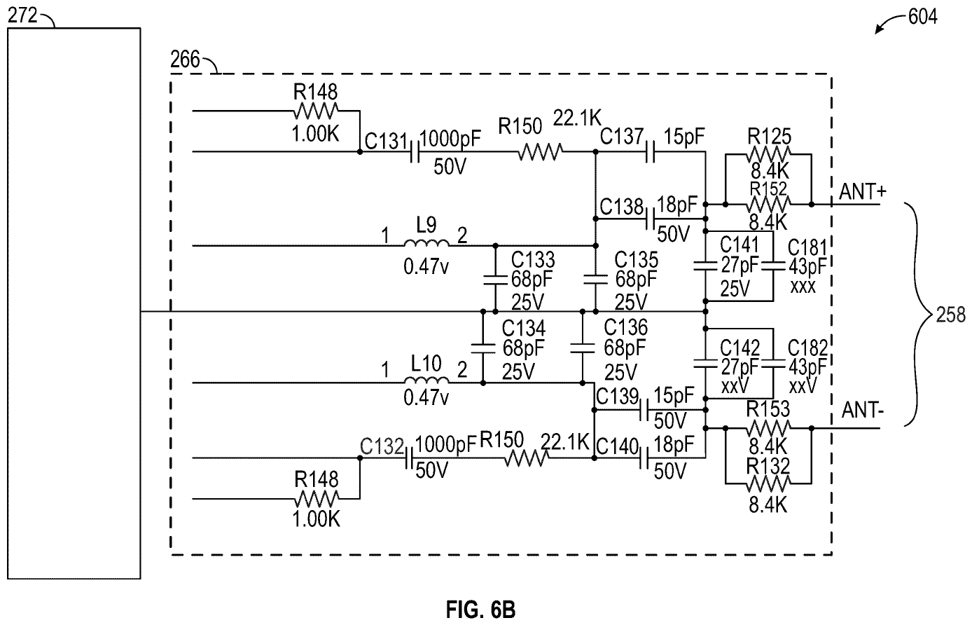

FIG. 6A illustrates a schematic of an electronic communications circuitry 602 of the pump assembly 160. A communications controller (or transceiver) 270 can be connected to the antenna 257. A matching network 262 can be interposed between the communications controller 270 and the antenna 257. The matching network 262 can be configured to match the output impedance of the antenna 257 (which can vary as the RF frequency varies) to the input impedance of the communications controller 270 at the desired communication protocol frequency (such as, about 13.56 MHz). The matching network 262 can include two sets of resistor pairs connected in parallel (R1 and R2; R3 and R4) and two sets of capacitor pairs connected in parallel (C2 and C2; C3 and C4). The capacitors of the matching network 262 can be used to counter the inductance of the antenna 257.

FIG. 6A also illustrates the antenna 268, which can facilitate communication with a remote computing device via another communication protocol. The antenna 268 can be a Bluetooth (or BLE) antenna. As described herein, the antenna 268 can be supported by the PCB 222. The antenna 268 can be a PIFA antenna, which can be positioned on the PCB 222. A matching network 264 can be interposed between the communications controller 270 and the antenna 268. The matching network 264 can be configured to match the output impedance of the antenna 268 (which can vary as the RF frequency varies) to the input impedance of the communications controller 270 at the desired communication protocol frequency (such as, about 2.4 GHz). The matching network 262 can include a z-network that includes an inductor L1 and two capacitors C6 and C7. In some cases, capacitor C6 can be omitted (for example, during manufacturing), and the matching network 264 becomes an L-network. The matching network 262 can serves to create a non-reactive antenna load for the communications controller 270. In some cases, the communications controller 270 can be used to operate the antenna 257 (which can be part of the passive NFC tag) and the antenna 268. For instance, the communications controller 270 can include two transceivers (one for the antenna 257 and the other for the antenna 268) on a single chip or integrated circuit. This can be advantageous to using separate communications controller for each of the antennas 257 and 268. For example, one or more of space, circuit complexity, power consumption, or cost can be reduced. In some implementations, separate communication controllers may be used.

FIG. 6B illustrates a schematic of an electronic communications circuitry 604 of the pump assembly 160. A communications controller (or transceiver) 272 can be connected to the antenna 258. A matching network 266 can be interposed between the communications controller 272 and the antenna 258. The antenna 258 can be part of the active NFC tag. The matching network 266 can be configured to match the output impedance of the antenna 258 (which can vary as the RF frequency varies) to the input impedance of the communications controller 272 at the desired communication protocol frequency (such as, about 13.56 MHz). The matching network 266 may be more complex than the matching network 262 because the antenna 258 can be configured for transmitting and receiving data (to support the active NFC tag). As is illustrated in FIGS. 6A-6B, different communication controllers can be utilized for the antennas 257 and 258.

### Cellular and GPS Connectivity

Any of the negative pressure wound therapy devices disclosed herein (such as, the pump assembly 160) can support one or more of cellular or GPS connectivity. Among other things, this can advantageously allow the device to send or receive data anywhere where cellular connectivity is present (irrespective of the range of the another communication protocol described herein) and allow tracking of the location of the device via GPS.

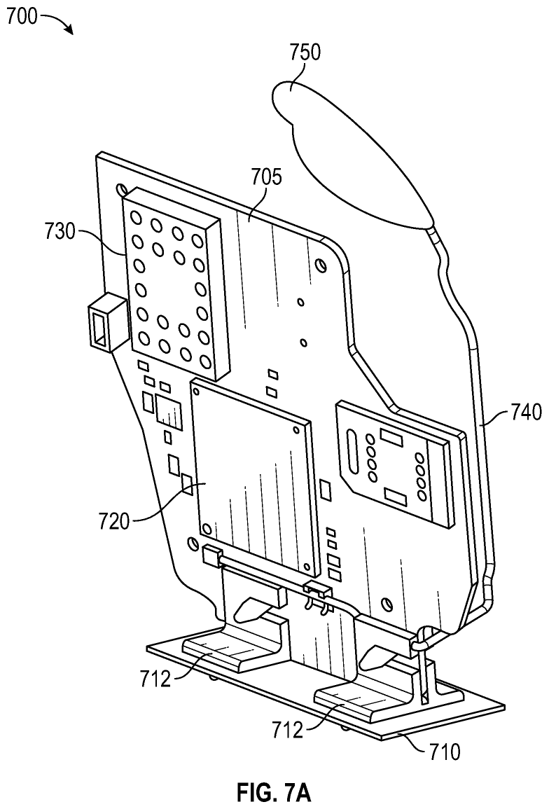

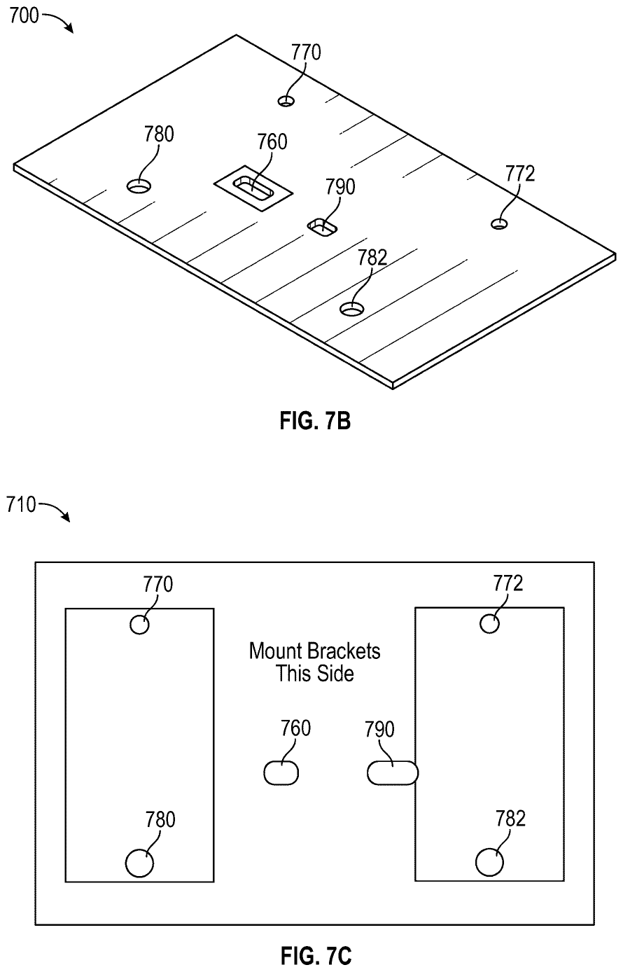

FIG. 7A illustrates a communications assembly 700 for cellular and GPS communications. The assembly 700 can be utilized by any of the negative pressure wound therapy devices disclosed herein, such as the pump assembly 160. The assembly 700 can include an antenna board 710 and a processor or communications board 705. One or more of the antenna board 710 or the communications board 705 can be a PCB. The communications board 705 can include a communications controller 720 (or transceiver). The communications board 705 can include a voltage regulator 730. One or more of the communications controller 720 or the voltage regulator 730 can be shielded.

The antenna board 710 can include wireless mobile communications antenna, such a single-, dual-, tri-, quad-or the like band antenna for communicating via 2G, 3G, LTE, 4G, 5G, or the like. The antenna can be referred to as a cellular antenna. The antenna board 710 can be mounted to the communications board 705 with mounting brackets 712. The communications assembly 700 can be electrically coupled via a path 740 to a GPS antenna 750. The antenna board 710 can be positioned in the bottom of the negative pressure wound therapy device (such as, the pump assembly 160). This positioning can advantageously shield the antenna board 710 (and the antenna) from interference (such as, electromagnetic interference) due to the presence and operation of internal electronic and electromechanical components (such as, the negative pressure source) and external noise (such as, electromagnetic interference, electrostatic discharge, or the like). Additionally or alternatively, such positioning may desirably afford additional space for increasing a size of the antenna to improve its performance (such as, improve the receive or transmit signal strength), as well as to enable the canister to function as a spacer to space the antenna from the ground or other surface on which the pump assembly is positioned. The antenna can be oriented to face downward (for example, toward the ground, floor, desk, bed, or other surface on which the pump assembly is positioned) rather than upward (for example, toward a ceiling or sky) or sideward (for example, toward side wall of a room) when the negative pressure wound therapy device is oriented for delivery of negative pressure therapy. This orientation can allow the antenna to reflect a communication signal (for example, a strongest signal or most of the energy of the signal received or output by the antenna) off the ground or another surface on which the device is positioned.

FIGS. 7B-7C illustrate the antenna board 710. FIG. 7B shows the top, perspective view of the board 710 illustrating holes 770, 772, 780, and 782 for aligning and attaching the mounting brackets and holes 760 and 790 for aligning and attaching the corresponding protrusions of the communications board 705. The hole 760 can includes a signal connector or connection between the antenna and a controller, such as the communications controller 720. FIG. 7C illustrates the bottom view of the antenna board 710. The mounting brackets 712 can be attached to the bottom side of the antenna board 710, which results in the top side of the antenna board 710 facing downward and away from the communications board 705 when the antenna board is attached or mounted to the communications board 705. In some cases, the mounting brackets 712 can be attached to the top side of the antenna board 710. The antenna can be oriented at any desired angle to the communications board 705, such as at 90 degrees, 80 degrees, 70 degrees, 60 degrees, and so on. In some implementations, desired orientation can be achieved by rotating or pivoting the mounting brackets 712.

In some implementations, the cellular antenna can be supported at least partially on an external surface of the pump assembly 160. This can overcome space restrictions inside the housing and allow the cellular antenna to be larger, allow multiple antennas to be used, or the like. Externally positioned cellular antenna can be supported by a portion of the exterior surface of the pump assembly 160 facing the canister. Advantageously, this can provide shielding from interference, as described herein. Externally positioned cellular antenna can be at least partially surrounded by an enclosure. The enclosure can include water resistant or waterproof material, such as rubber. Externally positioned cellular antenna can be connected to the communications circuit board located inside the housing (such as, the communications board 705) using a cable (for instance, a coaxial cable). In some cases, a bulkhead SMA connector can be used.

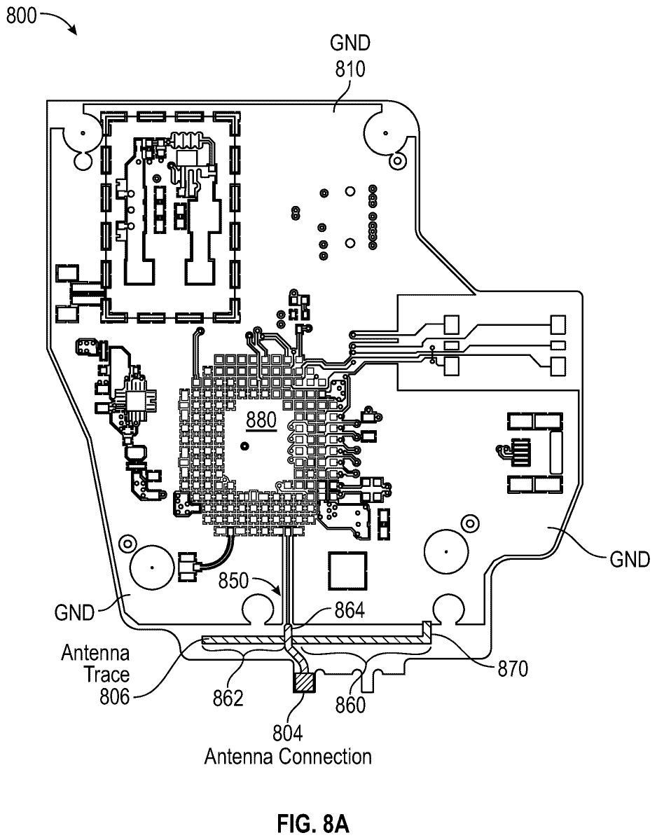

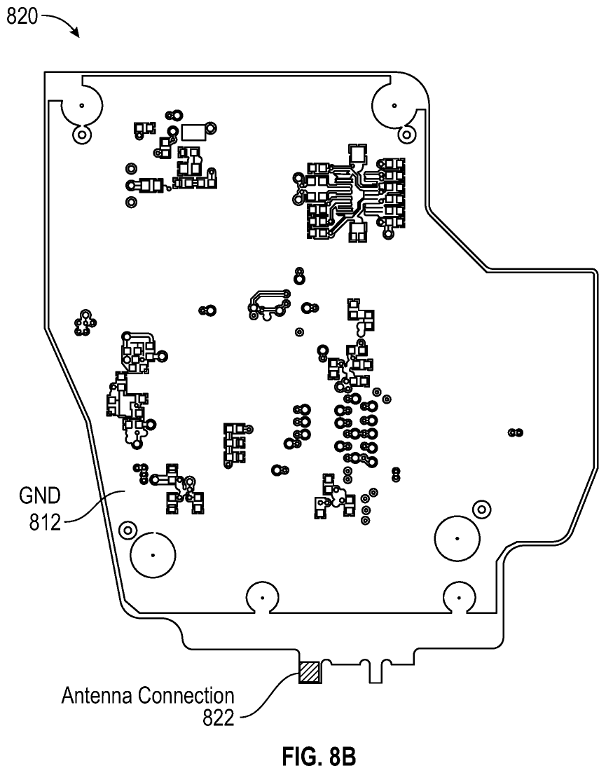

FIGS. 8A-8B illustrate a top layer or side 800 and bottom side 820 of a communications circuit board, such as the communications board 705. With reference to FIG. 8A, the top side 800 includes conductive portions (shown as darkened or shaded areas) and nonconductive portions or voids (shown as undarkened or white areas). The top side 800 can include a ground (GND) plane 810 and a connector or connection 804 between the antenna board 710 and the communications board 705. The connection 804 can provide a transmit signal feed from a controller (for example, the communication controller 720) and the antenna board 710 when antenna is transmitting (or is in a transmit mode). Connection 804 can provide a receive signal feed from the antenna board 710 to the controller when the antenna is receiving (or is in a receive mode). The connection 804 can, in some implementations, be the sole connection point for transmitting and receiving signals via the antenna board 710. Antenna trace 806, which includes first and second portions 860 and 862, is connected to the ground plane 810 at or near location 870. Location 870 can serve as shunt or ground connection of the antenna positioned on the antenna board 710. The trace 806 includes conductive material, such as copper, and can serve as a ground trace or ground plane for the antenna board 710. Connection between the trace 806 and the ground plane 810 of the communications board 705 can be accomplished with a shunt or another suitable component. Antenna trace 806 can be connected to the controller (located in area 880) via a feed path 850.

In certain implementations, the top side of the antenna board 710 (FIG. 7B) is placed facing down toward the ground and facing away from the communications board 705 when the antenna board is mounted to the communications board 705. Connection 760 can be located on the bottom side of the antenna board (FIG. 7C), which faces the communications board 705 when the antenna board is mounted to the communications board 705. In this configuration, the connection 804 on the communications board 705 faces connection 760 on the antenna board 710. A protrusion of the communications board 705 on which the connection 804 is positioned can be placed in the hole 760 of the antenna board 710. Electrical connection between the connection 804 and the hole 760 can be made, for example, using soldering or another suitable mechanism.

FIG. 8B illustrates a bottom side 820 of the communications circuit board of FIG. 8A. The bottom side 820 includes conductive portions (shown as darkened or shaded areas) and nonconductive portions or voids (shown as undarkened or white areas). The bottom side 820 can include a ground (GND) plane 812 and an antenna connector or connection 822. Connection 822 can be used as a mechanical connection for securing the antenna board 710. For instance, when the antenna board 710 is positioned top side facing downward as explained herein, because the connection 822 is positioned on the opposite side of the board with respect to the connection 804, more reliable or secure mechanical connection can be made by soldering, gluing, or using another suitable attachment a portion of the top surface of the antenna board (for example, area on the top side including and/or surrounding the hole 760) to the connection 822. In such instances, the connection 822 does not provide any electrical connectivity, but is used solely for mechanical support. As explained herein, the protrusion of the communications board 705 can be placed in the hole 760 of the antenna board 710 so that connection 822 is located proximal the top surface of the antenna board. Soldering the antenna board connection 760 on the opposite, bottom side of the antenna board 710 to the connection 804 can provide electrical connection and, optionally, additional mechanical support. In some cases, the locations of the antenna connection 822 and the connection 804 can be switched (for example, the antenna connection 822 can be placed on the top side 800) particularly when the antenna board is positioned top side facing upward away from the ground.

The antenna board 710 can be a PCB. The antenna board 710 can support the cellular antenna, which can be printed as one or more traces on the antenna board 710. For instance, the cellular antenna can be 4G/LTE or 5G antenna. In some cases, the cellular antenna can be a 4G antenna configured to transmit and receive in LTE frequency bands (such as, one or more of 1710-2155 MHz, 699-746 MHz, 824-294 MHz, 699-960 MHz, or 1710-2200 MHz). The cellular antenna can be an inverted-F antenna, such as a planar inverted-F antenna (PIFA). Such cellular antenna can be printed on a PCB (for instance, as one or more traces). The cellular antenna can be a microstrip antenna. As a result, the cellular antenna can be compact and inexpensive to manufacture. The antenna can be a quarter-wave length antenna. In certain implementations the antenna can be a monopole antenna, patch antenna, inverted-L antenna, or another suitable type of antenna.

In some cases, the cellular antenna may be located off a PCB, such as the antenna board 710. In such case, the antenna board 710 may not be present. The cellular antenna can be made out of metal, such as stamped metal, or be a flexible antenna, such as a flex circuit PCB antenna. Such cellular antenna can be positioned on the communications board 705 (such as, be soldered to the communications board 705) or be attached (such as, adhered) to the housing. In the latter case, the electrical connection between the cellular antenna and the communications board 705 can be made by attaching the antenna feed using a connector (such as, a spring connector or coaxial connector). In some implementations, the cellular antenna can be formed in the housing using laser direct structuring (LDS). This can advantageous to facilitate high volume, low cost manufacturing.

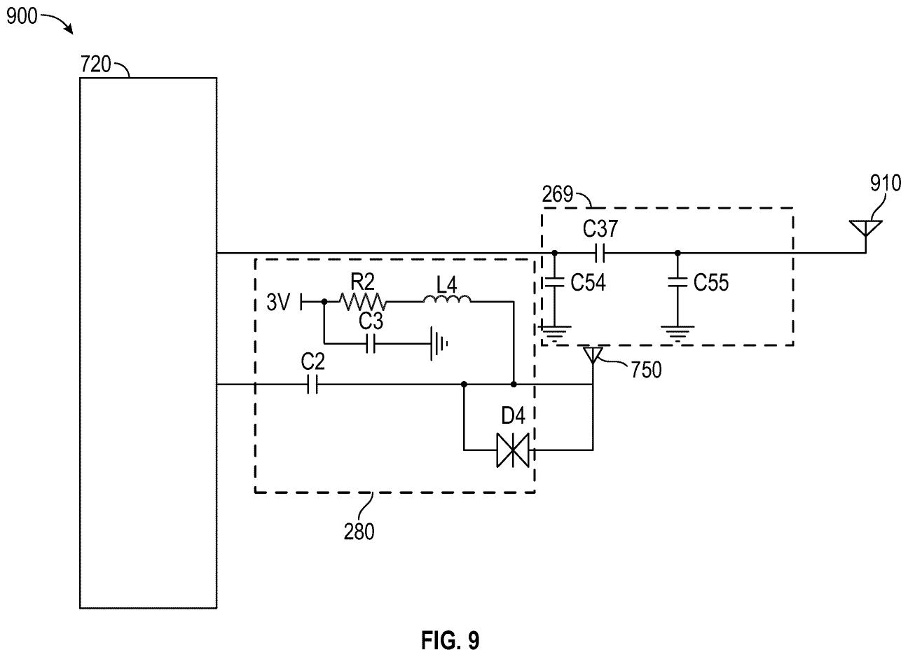

A matching network can be interposed between the communications controller of the communications board 705 (such as, the controller 720) and the cellular antenna. The matching network can be configured to match the output impedance of the cellular antenna (which can vary as the RF frequency varies) to the input impedance of the communications controller 720. FIG. 9 illustrates a schematic of electronic communications circuitry 900 that includes a matching network 269 interposed between the controller 720 and the cellular antenna 910. The matching network 269 can include a z-network that includes capacitors C37, C55, and C54. In some cases, one or more of the capacitors C54 or C55 may be omitted (for example, during manufacturing). When one of the capacitors C54 or C55 is omitted, the matching network 269 becomes an L-network. The matching network 269, which includes capacitors, can counter the impedance of the antenna 910.

The matching network 269 can extend the operational bandwidth of the cellular antenna, which can be quite wide for a 4G/LTE or 5G antenna. For example, 4G/LTE antenna may be required to have an operational bandwidth of 699 MHz (or less) to 2200 MHz (or more). The parallel capacitance (C54 and C55) of the matching network 269 can extend the operational bandwidth of the cellular antenna in the high band (such as, 1710-2170 MHz). The series capacitance (C37) can of the matching network 269 can extend the operational bandwidth of the cellular antenna in the low band (such as, 699-960 MHz).

The GPS antenna 750 can be connected to the controller 720 as is illustrated in FIG. 9. The GPS antenna 750 can be an active antenna and include an amplifier. The amplifier can be a low noise amplifier (LNA). The amplifier can be positioned near the GPS antenna 750, such as integrated into the same package as the GPS antenna. This can help avoid losses due to the use of longer connectors, as such losses can be detrimental to the performance because the intensity of GPS signals is quite small. The circuitry 280 can be configured to provide power to the amplifier of the GPS antenna (such as, 3V as shown in FIG. 9). Because power can be provided on the same pin as the GPS data, the circuitry 280 can include an inductor L4 that decouples the power signal from the GPS data (which can be a purely alternating current (AC) signal). The capacitor C3 and resistor R2 can facilitate removal of noise from the power signal as such noise may not be blocked by the inductor and may interfere with the GPS data. Diode D4 can protect the electronics from any voltage spikes. The diode D4 can be a transient voltage suppression diode. Capacitor C2 on the GPS data line can filter out noise (such as, direct current (DC) noise).

Advantageously, the controller 720 can control both the cellular antenna 910 and the GPS antenna 750. This can be preferable to using a separate controller for each of the antennas 910 and 750. In some implementations, separate controllers may be used.

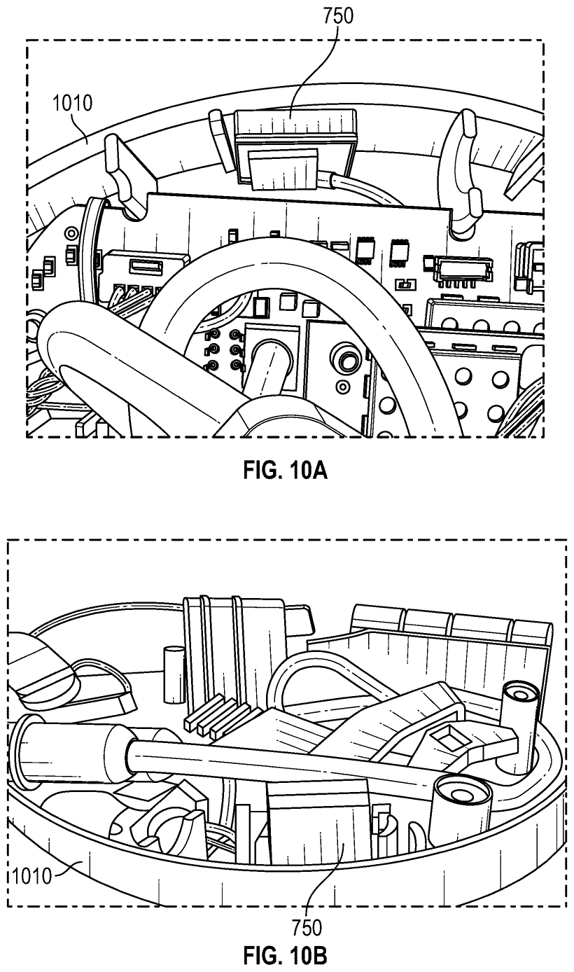

Further approaches to facilitating cellular and GPS communications are described in U.S. Patent Publication No. 2021/0030932, which is incorporated by reference in its entirety. FIGS. 10A-10B illustrate integration of a GPS antenna, such as the GPS antenna 750, into a negative pressure wound therapy device, such as the pump assembly 160. The GPS antenna can be positioned off a communications board, such as the communications board 705 (or the PCB 222). To facilitate better performance, the GPS antenna 750 can be located away from the electronic and electromechanical components of the negative pressure wound therapy device (such as, the pump), which may be sources of interference for the GPS antenna 750. As is shown in FIG. 10A, the GPS antenna 750 can be positioned at or near the top of the device inside the housing 1010. As is shown in FIG. 10B, the GPS antenna 750 can be positioned at or near one of the sides of the device inside the housing 1010. The GPS antenna 750 can be attached to the housing 1010 (such as, the top portion or side portion of the housing). For example, an adhesive can be used (such as, double-sided adhesive tape).

### Asset Management and Tracking

Any of the cellular antennas disclosed herein, such as the cellular antenna 910, can facilitate Internet of Things (IoT) or Machine-to-Machine (M2M) applications. In some instances, the cellular antenna can implement LTE Cat NB1, LTE Cat NB2, LTE Cat M1, LTE Cat 1, or 5G communications. Advantageously, the cellular antenna can provide support for applications requiring low power consumption (for instance, to allow operation for about 10 years (or less or more) without needing to replace or recharge the power supply). The cellular antenna can be used to provide efficient and effective asset management and tracking of negative pressure wound therapy devices and determination of the location of a patient. Additionally or alternatively, LTE can facilitate low latency (or rapid transmission), security, and privacy, provide backwards compatibility with 3G communications, provide forward compatibility with 5G communications, and facilitate making voice calls (which can allow the patient to contact the healthcare provider using the negative pressure wound therapy device). These benefits can be realized with a cellular antenna that supports LTE (such as, LTE Cat 1, LTE Cat M1, LTE Cat NB1, or LTE Cat NB2) or 5G. Such antenna can facilitate IoT or M2M applications. Unlike GPS, which may not work well inside a structure (such as, a building), asset management and tracking as well as location determination can be accurately performed indoors and outdoors using IoT or M2M. Unlike other more complex technologies that work indoors, such as WiFi or mesh networks that may require additional hardware and consume a lot of power, using IoT or M2M can be more cost-effective and promote device longevity.

The negative pressure wound therapy device can utilize such cellular antenna to transmit information regarding the device. The information can include one or more of identification of the negative pressure wound therapy device, date and time of communication with a base station (such as, a cell tower), location (which can be determined using triangulation), power source capacity level, device maintenance data, device calibration data, negative pressure wound therapy time, device standby time, device alarm data, device temperature data, whether the device has been moved (which can be determined using a motion sensor, such as an accelerometer or a gyroscope), determination whether the device has been used, or the like. The information can be transmitted periodically (such as, once per day, several times per day, once per several days, or the like). For instance, to conserve power, the negative pressure wound therapy device can periodically wake up, transmit the information, and return to low power (or sleep) mode. This can facilitate asset management and tracking of devices that may not yet be in use to provide therapy to a patient and, as result, may not be powered on for provision of negative pressure wound therapy (such as, devices at the factory or other storage, in transit, or in storage at the healthcare facility). The negative pressure wound therapy device can include a primary power source configured to power the device for provision of therapy and a secondary power source configured to power the cellular communications circuitry. As described herein, IoT or M2M applications can consume little power so that the secondary power source may not need to be recharged or replaced for 5 years (or less or more) or 10 years (or less or more).

In some cases, when the negative pressure wound therapy device is in transit on an aircraft, it may not be desirable to initiate or maintain cellular communications. The device can be configured to detect being on the aircraft and, in response to the detection, disable cellular communications until it has been detected that the device is no longer on the aircraft. The detection of being on (or off) the aircraft can be performed using an altitude meter (or altimeter) or a vibration sensor (such as, an accelerometer or a strain gauge) that can detect aircraft vibration.

Additional details of asset management and tracking and location determination are described in U.S. Pat. Nos. 9,737,649 and 10,744,239, each of which is incorporated by reference in its entirety.

### Other Variations

Although some embodiments describe negative pressure wound therapy, the systems, devices, and/or methods disclosed herein can be applied to other types of therapies usable standalone or in addition to TNP therapy. Systems, devices, and/or methods disclosed herein can be extended to any medical device, and in particular any wound treatment device. For example, systems, devices, and/or methods disclosed herein can be used with devices that provide one or more of ultrasound therapy, oxygen therapy, neurostimulation, microwave therapy, active agents, antibiotics, antimicrobials, or the like. Such devices can in addition provide TNP therapy. The systems and methods disclosed herein are not limited to medical devices and can be utilized by any electronic device.

Any of transmission of data described herein can be performed securely. For example, one or more of encryption, https protocol, secure VPN connection, error checking, confirmation of delivery, or the like can be utilized.

Any value of a threshold, limit, duration, etc. provided herein is not intended to be absolute and, thereby, can be approximate. In addition, any threshold, limit, duration, etc. provided herein can be fixed or varied either automatically or by a user. Furthermore, as is used herein relative terminology such as exceeds, greater than, less than, etc. in relation to a reference value is intended to also encompass being equal to the reference value. For example, exceeding a reference value that is positive can encompass being equal to or greater than the reference value. In addition, as is used herein relative terminology such as exceeds, greater than, less than, etc. in relation to a reference value is intended to also encompass an inverse of the disclosed relationship, such as below, less than, greater than, etc. in relations to the reference value.

Features, materials, characteristics, or groups described in conjunction with a particular aspect, embodiment, or example are to be understood to be applicable to any other aspect, embodiment or example described herein unless incompatible therewith. All of the features disclosed in this specification (including any accompanying claims, abstract and drawings), and/or all of the steps of any method or process so disclosed, can be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

While certain embodiments have been described, these embodiments have been presented by way of example only, and are not intended to limit the scope of protection. Indeed, the novel methods and systems described herein may be embodied in a variety of other forms. Furthermore, various omissions, substitutions and changes in the form of the methods and systems described herein may be made. Those skilled in the art will appreciate that in some embodiments, the actual steps taken in the processes illustrated and/or disclosed may differ from those shown in the figures. Depending on the embodiment, certain of the steps described above may be removed, others may be added. For example, the actual steps and/or order of steps taken in the disclosed processes may differ from those shown in the figure. Depending on the embodiment, certain of the steps described above may be removed, others may be added. For instance, the various components illustrated in the figures or described herein may be implemented as software and/or firmware on a processor, controller, ASIC, FPGA, and/or dedicated hardware. The software or firmware can include instructions stored in a non-transitory computer-readable memory. The instructions can be executed by a processor, controller, ASIC, FPGA, or dedicated hardware. Hardware components, such as controllers, processors, ASICs, FPGAs, and the like, can include logic circuitry. Furthermore, the features and attributes of the specific embodiments disclosed above may be combined in different ways to form additional embodiments, all of which fall within the scope of the present disclosure.

User interface screens illustrated and described herein can include additional and/or alternative components. These components can include menus, lists, buttons, text boxes, labels, radio buttons, scroll bars, sliders, checkboxes, combo boxes, status bars, dialog boxes, windows, and the like. User interface screens can include additional and/or alternative information. Components can be arranged, grouped, displayed in any suitable order.

Conditional language used herein, such as, among others, “can,” “could”, “might,” “may,” “e.g.,” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Further, the term “each,” as used herein, in addition to having its ordinary meaning, can mean any subset of a set of elements to which the term “each” is applied. Additionally, the words “herein,” “above,” “below,” and words of similar import, when used in this application, refer to this application as a whole and not to any particular portions of this application.

Conjunctive language, such as the phrase “at least one of X, Y and Z,” unless specifically stated otherwise, is to be understood with the context as used in general to convey that an item, term, etc. may be either X, Y, or Z, or a combination thereof. Thus, such conjunctive language is not generally intended to imply that certain embodiments require at least one of X, at least one of Y and at least one of Z to each be present.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain embodiments, the terms “generally parallel” and “substantially parallel” refer to a value, amount, or characteristic that departs from exactly parallel by less than or equal to 15 degrees, 10 degrees, 5 degrees, 3 degrees, 1 degree, or 0.1 degree.

Unless otherwise explicitly stated, articles such as “a” or “an” should generally be interpreted to include one or more described items. Accordingly, phrases such as “a device configured to” are intended to include one or more recited devices. Such one or more recited devices can also be collectively configured to carry out the stated recitations.

Although the present disclosure includes certain embodiments, examples and applications, it will be understood by those skilled in the art that the present disclosure extends beyond the specifically disclosed embodiments to other alternative embodiments and/or uses and obvious modifications and equivalents thereof, including embodiments which do not provide all of the features and advantages set forth herein. Accordingly, the scope of the present disclosure is not intended to be limited by the specific disclosures of preferred embodiments herein, and may be defined by claims as presented herein or as presented in the future.

1-30. (canceled)

31. A negative pressure wound therapy device comprising:
a housing;
a negative pressure source supported by the housing, the negative pressure source configured to provide negative pressure to a wound covered by a wound dressing;
an electronic control circuitry supported by the housing, the electronic control circuitry configured to operate the negative pressure source to provide negative pressure in accordance with a set of negative pressure wound therapy parameters;
a first antenna positioned in first portion of the housing that comprises a top portion of the housing or a side portion of the housing, the first antenna configured to facilitate wireless communications with a remote computing device, and the electronic control circuitry configured to utilize the first antenna to exchange pairing data with the remote computing device using a first communication protocol to allow exchange of data between the remote computing device and the negative pressure wound therapy device;
a second antenna positioned in a second portion of the housing configured to support a canister that is configured to store exudate aspirated from the wound as a result of provision of negative pressure to the wound, the second portion of the housing different from the first portion of the housing, the second antenna configured to facilitate wireless communications with the canister when the canister is removably attached to the second portion of the housing, the electronic control circuitry configured to utilize the second antenna to receive, from the canister, canister data comprising one or more of a fill status of the canister, an indication that the canister is full, identification data, configuration data, date of manufacture of the canister, or date of first use of the canister, and the electronic control circuitry further configured to use the canister data to control provision of negative pressure to the wound; and
a third antenna configured to facilitate wireless communications with the remote computing device using a second communication protocol that is different from the first communication protocol, the electronic control circuitry configured to utilize the third antenna to transmit data comprising at least one of a location of the housing, logs corresponding to provision of negative pressure, or alarms caused by provision of negative pressure.

32. The negative pressure wound therapy device of claim 31, wherein the electronic control circuitry is further configured to utilize the third antenna to receive data comprising at least one of data for tracking the location of the housing, data for adjusting the set of negative pressure wound therapy parameters, or data for upgrading software or firmware.

33. The negative pressure wound therapy device of claim 31, wherein the second communication protocol permits communication over a longer distance than the first communication protocol.

34. The negative pressure wound therapy device of claim 31, wherein the first communication protocol comprises a near-field protocol and the second communication protocol comprises Bluetooth or Bluetooth low energy (BLE) protocol.

35. The negative pressure wound therapy device of claim 31, wherein the electronic control circuitry is configured to non-simultaneously transmit or receive data using the first or second antenna.

36. The negative pressure wound therapy device of claim 31, wherein the electronic control circuitry is configured to determine that the canister is connected to the housing responsive to receiving the canister data.

37. The negative pressure wound therapy device of claim 36, wherein the electronic control circuitry is configured to disallow activation of the negative pressure source responsive to determining that the canister is not connected to the housing.

38. The negative pressure wound therapy device of claim 31, wherein the electronic control circuitry further comprises:
a first transceiver connected to the first and third antennas;
a second transceiver connected to the second antenna;
a first matching circuitry interposed between the first transceiver and the first antenna, the first matching circuitry configured to match impedance of the first antenna with a first input impedance of the first transceiver;
a second matching circuitry interposed between the first transceiver and the third antenna, the second matching circuitry configured to match impedance of the third antenna with a second input impedance of the first transceiver; and
a third matching circuitry interposed between the second transceiver and the second antenna, the third matching circuitry configured to match impedance of the second antenna with an input impedance of the second transceiver.

39. The negative pressure wound therapy device of claim 38, wherein the first matching circuitry comprises at least one capacitor and at least one resistor, and wherein the third matching circuitry comprises at least one capacitor, at least one inductor, and at least one resistor.

40. The negative pressure wound therapy device of claim 31, wherein the first antenna is positioned in the top portion of the housing, the second antenna is positioned in a bottom portion of the housing, and the third antenna is positioned in the top portion of the housing proximate to the first antenna.

41. A negative pressure wound therapy device comprising:
a housing;
a negative pressure source supported by the housing, the negative pressure source configured to provide negative pressure to a wound covered by a wound dressing;
an electronic control circuitry supported by the housing, the electronic control circuitry configured to operate the negative pressure source to provide negative pressure in accordance with a set of negative pressure wound therapy parameters; and
a communication circuitry supported by the housing and comprising:
a cellular antenna located in a first portion of the housing and configured to facilitate wireless communication using long term evolution (LTE) communication protocol, the first portion of the housing comprising a bottom portion of the housing;
a global positioning system (GPS) antenna located in a second portion of the housing comprising a top portion of the housing or a side portion of the housing, the second portion of the housing being different from the first portion of the housing;
a transceiver configured to utilize the cellular antenna and the GPS antenna to transmit data comprising at least one of a location of the housing, logs corresponding to provision of negative pressure, or alarms caused by provision of negative pressure; and
a matching circuitry interposed between the cellular antenna and the transceiver, the matching circuitry configured to match impedance of the cellular antenna with an input impedance of the transceiver.

42. The negative pressure wound therapy device of claim 41, wherein the transceiver if further configured to utilize the cellular antenna and the GPS antenna to receive data comprising at least one of data for tracking the location of the housing, data for adjusting the set of negative pressure wound therapy parameters, or data for upgrading software or firmware using the cellular antenna and the GPS antenna.

43. The negative pressure wound therapy device of claim 41, wherein the matching circuitry comprises a w-network or an L-network with a plurality of capacitors.

44. The negative pressure wound therapy device of claim 41, further comprising a first power source configured to provide power at least to the negative pressure source and a second power source configured to provide power at least to the transceiver, wherein the transceiver is configured to transmit location information using an LTE or 5G communication protocol.

45. The negative pressure wound therapy device of claim 44, wherein the transceiver is configured to transmit location information using the LTE or 5G communication protocol when the first power source does not provide power to the negative pressure source or any other component of the negative pressure wound therapy device.

46. The negative pressure wound therapy device of any of claims 44, wherein the transceiver is configured to transmit location information using LTE Cat M1.

47. The negative pressure wound therapy device of claim 41, wherein the electronic control circuitry is configured to disable the cellular antenna responsive to detecting that the negative pressure wound therapy device is being carried on an aircraft.

48. The negative pressure wound therapy device of claim 47, wherein the electronic control circuitry is configured to detect that the negative pressure wound therapy device is being carried on an aircraft based on monitoring at least one of an altitude or vibration.

49. The negative pressure wound therapy device of claim 41, wherein the GPS antenna comprises an amplifier, and wherein the electronic control circuitry further includes circuitry configured to provide power to the amplifier using the same connection of the GPS antenna on which data is communicated to the transceiver.

50. The negative pressure wound therapy device of claim 41, wherein the transceiver is positioned on a first printed circuit board (PCB) supported by the housing, and wherein the cellular antenna is positioned on a second PCB electrically connected to the first PCB by a single connector of the first PCB, the single connector further providing mechanical support for the second PCB.
